(19) United States
(12) Patent Application Publication
(10) Pub. No.: US 2003/0228361 A1
(43) Pub. Date: Dec. 11, 2003

(54) Sustained release metoprolol formulations

(76) Inventors: Anand R. Baichwal, Wappingers Fall, NY (US); Paul Woodcock, Brookfield, CT (US); Pradeepkumar P. Sanghvi, New Fairfeild, CT (US); Dean M. Dinicola, Prospect, CT (US); Troy W. McCall, Germantown, TN (US)

Correspondence Address: DAVIDSON, DAVIDSON & KAPPEL, LLC, 14th Floor, 485 Seventh Avenue, New York, NY, 10018, US

(21) Appl. No.: 10/408,739

(22) Filed: Apr. 7, 2003

Related U.S. Application Data
(60) Provisional application No. 60/370,460, filed on Apr. 5, 2002.
(60) Provisional application No. 60/442,228, filed on Jan. 24, 2003.

(51) Int. Cl. A61K 9/22; A61K 31/137

(52) U.S. Cl. 424/468; 514/651

(57) ABSTRACT

The present invention relates to a sustained release oral dosage forms containing a therapeutically effective amount of metoprolol tartrate, methods of preparing such formulations, and to methods of treatment utilizing such formulations.

This application claims priority from U.S. Provisional Application No. 60/370,460, filed Apr. 5, 2002, and U.S. Provisional Application No. 60/442,228, filed Jan. 24, 2003, the disclosures of which are hereby incorporated by reference in their entireties.

### FIELD OF THE INVENTION

The present invention relates to sustained release oral dosage forms containing a therapeutically effective amount of metoprolol tartrate. The present invention is further related to methods of preparing such formulations, and to methods of treatment utilizing such formulations.

### BACKGROUND OF THE INVENTION

The advantages of controlled release products are well known in the pharmaceutical field and include the ability to maintain a desired blood level of a medicament over a comparatively longer period of time while increasing patient compliance by reducing the number of administrations necessary to achieve the same. These advantages have been attained by a wide variety of methods.

For example, different hydrogels have been described for use in controlled release medicines, some of which are synthetic, but most of which are semi-synthetic or of natural origin. A few contain both synthetic and non-synthetic material. However, some of the systems require special process and production equipment, and in addition some of these systems are susceptible to variable drug release.

Oral controlled release delivery systems should ideally be adaptable so that release rates and profiles can be matched to physiological and chronotherapeutic requirements.

While many controlled and sustained-release formulations are already known, certain soluble to highly soluble drugs present formulation difficulties when included in such formulation. An example of such a highly soluble drug is metoprolol tartrate.

There have been a number of patents in the prior art which relate to controlled release metoprolol formulations. For example, U.S. Pat. No. 5,169,638 describes a buoyant controlled release pharmaceutical formulation in the form of a powder filled capsule in which an active ingredient of a basic character exhibits a pH-independent controlled release. The powder comprises the active agent, which may be metoprolol, a water-soluble salt of polyuronic acid, a pH-independent hydrocolloid gelling agent (e.g., hydroxypropylmethylcellulose, methylcellulose or hydroxypropylcellulose), and a binder (HPMC). The formulation is free of calcium ion and carbon dioxide producing material and is said to float gastric juices so that it will have extended residence time in the stomach.

U.S. Pat. No. 4,792,452 describes controlled release pharmaceutical compositions which are said to provide pH-independent release for a basic drug such as metoprolol. The formulations include a pH-dependent polymer which is a salt of alginic acid, a pH-independent hydrocolloid gelling agent and a binder. The salt of the alginic acid is preferably sodium alginate or potassium alginate. The weight ratio of the alginic acid salt to the hydrocolloid gelling agent is all within the range 0.1:1 to 10:1, and the formulation is free of calcium ion and carbon dioxide-producing material.

U.S. Pat. No. 4,957,745 also describes a controlled release metoprolol. The preparation includes a plurality of beads comprising metoprolol coated with a polymeric membrane comprising ethylcellulose with or without hydroxypropylmethylcellulose.

U.S. Pat. No. 4,871,549 describes a time controlled explosion system comprising metoprolol, a swelling agent such as a low substituted hydroxypropylcellulose, sodium starch glycolate or carboxymethylcellulose sodium, coated with a water-insoluble coating material so that drug release is caused by the explosion of the membrane after a definite time period.

U.S. Pat. No. 5,081,154 is directed to metoprolol succinate in an oral composition coated with an anionic polymer soluble at pH over 5.5 and a water insoluble quaternary ammonium substituted acrylic polymer.

Further, U.S. Pat. Nos. 5,399,358 and 5,399,362 disclose a sustained release oral solid dosage form of metoprolol which includes a sustained release excipient including a gelling agent, an inert pharmaceutical diluent, and a cationic cross-linking agent. The formulation provides release of metoprolol for at least about 24 hours.

All documents cited herein, including the foregoing, are incorporated by reference in their entireties for all purposes.

A metoprolol formulation marketed in the United Kingdom is Betaloc® S.A., which contains 200 mg of metoprolol tartrate in a controlled release matrix.

Currently, metoprolol is available as 50 mg, 100 mg and 200 mg extended release tablets in the United States and is marketed under the name Toprol XL® from AstraZeneca. Toprol XL® tablets contain the succinate salt of metoprolol (equivalent to 50 mg, 100 mg and 200 mg of the tartrate salt) and are indicated for the treatment of hypertension. Toprol XL® tablets comprise a multiple unit system containing metoprolol succinate in a multitude of controlled release pellets. These tablets may be dosed once daily. Studies have shown that formulations similar to those of Toprol XL®, containing metoprolol succinate in a multitude of controlled release pellets, has a more sustained time profile of B₁-blockade at steady-state than formulations similar to those of Betaloc® S.A., containing metoprolol tartrate in a controlled release matrix. See, e.g., Berend Oosterhuis, PhD, et al., “A Pharmacokinetic and Pharmacodynamic Comparision of Metoprolol CR/ZOK with a Conventional Slow Release Preparation,” J Clin. Pharmacol., 1990:30:533-538. Additionally, these studies have shown that metoprolol succinate in an extended release form similar to Toprol XL® had mean and individual plasma concentration-time profiles that were said to be smoother than the profiles of formulations such as those of Betaloc® S.A. Further, for formulations containing metoprolol succinate in a controlled release form similar to Toprol XL<, the value of Cₘₐₓ was significantly lower, the Cₘᵢₙ was higher, the Tₘₐₓ value tended to be longer, and the time during which the metoprolol plasma concentration exceeded 75% of Cₘₐₓ was significantly longer versus formulations, similar to those of Betaloc® S.A. containing metoprolol tartrate in a controlled release matrix.

Accordingly, there exists a need in the art to provide a sustained release oral dosage form that provides for the sustained release of metoprolol tartrate suitable for once-a-day administration.

### OBJECTS AND SUMMARY OF THE INVENTION

It is an object of the present invention to provide an oral sustained release formulation of metoprolol tartrate suitable for once-a-day administration.

It is a further object of certain embodiments of the present invention to provide oral solid sustained release formulations which release metoprolol tartrate over a time period of at least about 24 hours, when the formulations are exposed to an environmental fluid (e.g., the gastrointestinal tract).

It is a further object of certain embodiments of the present invention to provide a sustained release oral dosage form comprising metoprolol tartrate which provides for improved pharmacokinetic parameters than prior metoprolol tartrate sustained release formulations.

It is a further object of certain embodiments of the present invention to provide methods for preparing sustained release metoprolol tartrate formulations which may be administered to patients on a once-a-day basis, or a desired longer time interval.

It is a further object of certain embodiments of the present invention to provide a method of preparing sustained release metoprolol tartrate formulations which result in improved flow and tableting characteristics as compared to prior methods of preparing metoprolol tartrate formulations.

The above-mentioned objects and others are achieved by virtue of the present invention, which is directed in part to a sustained release oral solid dosage form comprising a therapeutically effective amount of metoprolol tartrate, and a sustained release excipient, said oral dosage form providing a mean Cₘₐₓ from about 10 ng/ml to about 40 ng/ml per 100 mg metoprolol tartrate; said dosage form providing a therapeutic effect for about 24 hours after oral administration.

In certain preferred embodiments, the sustained release oral dosage forms of the present invention provide a mean Cₘₐₓ from about 15 ng/ml to about 30 ng/ml per 100 mg metoprolol tartrate.

In certain embodiments, the sustained release oral dosage forms of the present invention provide a mean Cₘₐₓ from about 40 ng/ml to about 90 ng/ml per 200 mg metoprolol tartrate.

In certain embodiments, the sustained release oral dosage forms of the present invention provide a mean Cₘₐₓ from about 5 ng/ml to about 30 ng/ml per 50 mg metoprolol tartrate.

In certain embodiments, the sustained release oral dosage forms of the present invention provide a mean Cₘₐₓ from about 2 ng/ml to about 15 ng/ml per 25 mg metoprolol tartrate.

In certain embodiments, the sustained release oral dosage forms of the present invention provide a mean Cₘₐₓ at steady state of from about 5 ng/ml to about 30 ng/ml per 50 mg metoprolol tartrate, preferably from about 10 ng/ml to about 25 ng/ml per 50 mg metoprolol tartrate.

In certain embodiments, the sustained release oral dosage forms of the present invention provide a mean Cₘₐₓ at steady state of from about 4 ng/ml to about 20 ng/ml per 25 mg metoprolol tartrate, preferably from about 6 ng/ml to about 15 ng/ml per 25 mg metoprolol tartrate.

In certain embodiments, when the dosage form contains 100 mg metoprolol tartrate, the sustained release oral dosage form provides a mean Cₘₐₓ of metoprolol from about 10 ng/ml to about 40 ng/ml, preferably from about 15 ng/ml to about 30 ng/ml. In certain embodiments, when the dosage form contains 200 mg metoprolol tartrate, the sustained release oral dosage form provides a mean Cₘₐₓ of metoprolol from about 40 ng/ml to about 90 ng/ml. In certain embodiments, when the dosage form contains 50 mg metoprolol tartrate, the sustained release oral dosage form provides a mean Cₘₐₓ of metoprolol from about 5 ng/ml to about 30 ng/ml. In certain embodiments, when the dosage form contains 25 mg metoprolol tartrate, the sustained release oral dosage form provides a mean Cₘₐₓ of metoprolol from about 2 ng/ml to about 15 ng/ml.

In certain embodiments, when the dosage form contains 25 mg metoprolol tartrate, the sustained release oral dosage form provides a mean Cₘₐₓ at steady state of metoprolol from about 4 ng/ml to about 20 ng/ml, preferably from about 6 ng/ml to about 15 ng/ml. In certain embodiments, when the dosage form contains 50 mg metoprolol tartrate, the sustained release oral dosage form provides a mean Cₘₐₓ at steady state of metoprolol from about 5 ng/ml to about 30 ng/ml, preferably from about 10 ng/ml to about 25 ng/ml.

In certain embodiments, the present invention is further directed to a sustained release oral solid dosage form comprising a therapeutically effective amount of metoprolol tartrate and sustained release excipient; wherein the dissolution rate in-vitro of the dosage form, when measured by the USP Apparatus Type III at 37° C.±0.5 in 250 ml (per dissolution vessel) at 15 dpm and 0.1 M pH 7.5 is preferably as follows: from 0% to about 10% metoprolol tartrate released at about 1 hour; from about 5% to about 30% metoprolol tartrate released at about 3 hours; from about 20% to about 60% metoprolol tartrate released at about 6 hours; from about 30% to about 70% metoprolol tartrate released at about 8 hours; greater than about 50% metoprolol tartrate release at about 16 hours; and greater than about 80% metoprolol tartrate release at about 24 hours.

In certain embodiments, the present invention is further directed to a sustained release oral solid dosage form for absorption of a therapeutically active medicament in the gastrointestinal tract, said therapeutically active medicament comprising an effective amount of metoprolol tartrate; and a sustained release excipient comprising a gelling agent comprising a heteropolysaccharide gum and a homopolysaccharide gum capable of cross-linking said heteropolysaccharide gum when exposed to an environmental fluid, said oral dosage form providing a mean Cₘₐₓ from about 10 ng/ml to about 40 ng/ml per 100 mg metoprolol tartrate; said dosage form providing a therapeutic effect for about 24 hours after oral administration.

In certain embodiments, the sustained release oral dosage form of the present invention comprises a therapeutically effective amount of metoprolol tartrate, and a sustained release excipient comprising a heteropolysaccharide gum and a homopolysaccharide gum capable of cross-linking said heteropolysaccharide gum when exposed to an environmental fluid; a cellulose derivative such as, e.g., an alkylcellulose, hydroxyalkylcellulose, hydroxypropylalkylcellulose, or mixtures thereof; an inert diluent selected from, e.g., a monosaccharide, a disaccharide, a polyhydric alcohol, or mixtures thereof; and an effective amount of a pharmaceutically acceptable water-soluble cationic cross-linking agent; said dosage form providing a mean Cₘₐₓ from about 10 ng/ml to about 40 ng/ml per 100 mg metoprolol tartrate administered over a 24 hour period, said dosage form preferably providing a sustained release of the medicament for about 24 hours, when the dosage form is exposed to an environmental fluid.

In certain preferred embodiments of the present invention, the sustained release oral dosage form comprising the metoprolol tartrate and sustained release excipient, is overcoated with a coating that in addition to the sustained release excipient of the sustained release oral dosage form controls the release of the metoprolol tartrate from the formulation, such that blood levels of active ingredient are maintained within the therapeutic range over an extended period of time, and providing a mean Cₘₐₓ from about 10 ng/ml to about 40 ng/ml per 100 mg metoprolol tartrate administered over a 24 hour period.

In certain preferred embodiments, the sustained release oral dosage form of the present invention further provides a mean Tₘₐₓ at from about 2.5 to about 20 hours after oral administration of the dosage form. In certain preferred embodiments, the sustained release oral dosage form of the present invention further provides a mean Tₘₐₓ at from about 6 to about 16 hours after oral administration of the dosage form.

In certain embodiments of the present invention, the Cₘₐₓ values achieved by the dosage forms of the present invention are based dose proportional formulations. In certain embodiments, the dosage forms of the present invention are dose proportional or substantially dose proportional.

In certain embodiments of the present invention, the pharmacokinetic values are based on administration to a human subject. Alternatively, the pharmacokinetic values are based on administration to a human patient.

In certain preferred embodiments of the invention wherein a gum is included in a sustained release excipient, the gum is included in an amount from about 10% to about 60%, and more preferably from about 10% to about 50%, by weight of the final product. The drug to gum ratio may be, e.g., from about 1:0.5 to about 1:7. More preferably, the drug to gum ratio is from about 1:0.7 to about 1:6.

In certain preferred embodiments, when the sustained release excipient comprises a gelling agent, the sustained release excipient further comprises an effective amount of an ionizable gel strength enhancing agent to obtain a desirable increased gel strength due to cross-linking of the gelling agent in the sustained release excipient.

In certain preferred embodiments, the sustained release excipient further comprises a hydrophobic material in an amount effective to slow the hydration of the gums without disrupting the hydrophilic matrix formed by the heterodisperse polysaccharide when the formulation is exposed to fluids in an environment of use.

In certain embodiments, the present invention is further directed to a sustained release excipient for the sustained release of an active agent comprising from about 20% to about 60% of a gelling agent by weight of said sustained release excipient, said gelling agent consisting of a heteropolysaccharide gum and a homopolysaccharide gum; from about 1% to about 20% of an ionizable gel strength enhancing agent by weight of said sustained release excipient; and from about 6% to about 60% of mannitol by weight of the sustained release excipient.

In certain preferred embodiments, the present invention is directed to a method of preventing or reducing a mallaird-type reaction in a metoprolol tartrate sustained release oral dosage form comprising preparing said sustained release oral dosage form by combining a therapeutically effective amount of metoprolol tartrate with a sustained release excipient that provides for the sustained release of said metoprolol tartrate, and including in said dosage form an effective amount of mannitol to prevent or reduce the degradation of said metoprolol tartrate.

In certain preferred embodiments, the present invention is further directed to a sustained release oral dosage form comprising metoprolol tartrate in an amount of from about 12.5 mg to about 400 mg dispersed in a matrix comprising (i) a gelling agent said gelling agent in an amount of from about 10% to about 60% by weight of the dosage form, (ii) an inert pharmaceutical diluent in an amount of from about 5% to about 40% by weight of the dosage form, and (iii) an ionizable gel strength enhancing agent in an amount of from about 0.5% to about 16% by weight of the dosage form; a hydrophobic coating coated over said matrix in an amount of from about 1% to about 20% by weight of the dosage form; wherein the formulation provides for the sustained release of the metoprolol tartrate and is suitable for once-a-day administration.

In certain preferred embodiments, the present invention is further directed to a sustained release oral dosage form comprising a matrix comprising metoprolol tartrate in an amount of from about 12.5 mg to about 400 mg dispersed in a sustained release excipient comprising (i) locust bean gum in an amount of 5% to about 30% by weight of the oral dosage form and (ii) xanthan gum in an amount from about 5% to about 30% by weight of the oral dosage form, (iii) mannitol in an amount of from about 5% to about 40% by weight of the oral dosage form, and (iv) calcium sulfate dihydrate in an amount of about 0.5% to about 16% by weight of the oral dosage form; and a hydrophobic coating coated over said matrix in an amount of from about 1% to about 20% by weight of the oral dosage form; wherein the formulation provides for the sustained release of the metoprolol tartrate and is suitable for once-a-day administration.

In certain preferred embodiments, the present invention is further directed to a sustained release tablet formulation comprising: a matrix core composition comprising metoprolol tartrate in an amount of from about 12.5 mg to about 400 mg; a cellulose derivative selected from the group consisting of an alkylcellulose, hydroxyalkylcellulose, hydroxypropylalkylcellulose, or mixtures thereof; and a sustained release excipient comprising a gelling agent in an amount of about 10% to about 60% by weight of the formulation; an inert diluent in an amount of from about 5% to about 40% by weight of the formulation; and an ionizable gel strength enhancing agent in an amount of from about 0.5% to about 16% by weight of the formulation; and a coating over said core comprising a hydrophobic material in an amount of from about 2% to about 15% by weight of the formulation; wherein the formulation provides for the sustained release of the metoprolol tartrate and is suitable for once-a-day administration.

In one embodiment, a sustained release tablet formulation of the present invention comprises about 31% by weight of metoprolol tartrate; about 45% by weight of a sustained release excipient comprising xanthan gum, locust bean gum, calcium sulfate dihydrate, and mannitol; about 3% by weight hydroxypropylmethylcellulose; about 4% by weight talc; about 2% by weight sodium stearyl fumarate; about 9 to about 12% by weight hydrophobic coating material; and about 3% by weight of a color coating material; and the formulation provides for the sustained release of the metoprolol tartrate.

In another embodiment, a sustained release tablet formulation of the present invention comprises about 32% by weight of metoprolol tartrate; about 48% by weight of a sustained release excipient comprising xanthan gum, locust bean gum, calcium sulfate dihydrate, and mannitol; about 4% by weight hydroxypropylmethylcellulose; about 4% by weight talc; about 2% by weight sodium stearyl fumarate; about 8% by weight hydrophobic coating material; and about 3% by weight of a color coating material; and the formulation provides for the sustained release of the metoprolol tartrate.

In certain embodiments, the present invention is also related to a method for providing a sustained release oral dosage form of metoprolol tartrate, comprising preparing a sustained release excipient by (1) dry blending a heteropolysaccharide gum and a homopolysaccharide gum capable of cross-linking said heteropolysaccharide gum when exposed to an environmental fluid, together with a pharmaceutically acceptable inert diluent in desired proportions and an optional ionizable gel strength enhancing agent; (2) wet granulating the mixture; (3) drying the resultant granulate; and (4) milling the dried granulate to obtain a sustained release excipient having a desired particle size. Thereafter, the sustained release excipient is (5) blended with metoprolol tartrate and optional additional diluent(s) and excipients(s) as desired, and (6) the mixture of sustained release excipient and metoprolol tartrate is spray granulated with a solution or suspension preferably of a cellulose derivative, e.g., alkylcellulose, hydroxyalkylcellulose, hydroxyalkylalkylcellulose, or mixtures thereof, and (7) the resultant granulate is dried. Next, any (8) additional inert excipients are added (e.g., a lubricant, glidant, etc.) and the resultant mixture is then, e.g., (9) compressed into tablets; thereafter in certain embodiments, the tablets are then (10) overcoated with a coating comprising a hydrophobic material.

In certain embodiments, the mixture of the sustained release excipient is granulated with a solution of a hydrophobic material in an amount sufficient to slow the hydration of the gums without disrupting the same prior to mixing the sustained release excipient with the metoprolol tartrate.

In preferred embodiments set forth herein and in the appended claims, the formulations of the present invention do not require the inclusion of a pH-modifying agent, e.g., as described in the assignee's International Patent Publication WO 01/22940.

In preferred embodiments, the formulations of the present invention are cardioselective. Preferably the present invention is further directed to a method or providing cardioselective anti-hypertensive therapy to a patient by administering a sustained release metoprolol tartrate dosage from of the present invention to a patient in need of such treatment.

The present invention is further related to a method of treating hypertension, angina, and/or heart failure, comprising orally administering a sustained release metoprolol tartrate dosage form of the present invention to a patient in need of such treatment, thereby providing therapeutically effective blood levels of the medicament for at least about 24 hours, after administration. In certain embodiments, the present invention is further related to a method of reducing blood pressure, comprising orally administering a sustained release metoprolol tartrate dosage form of the present invention to a human patient or human subject.

Preferably, the low Cₘₐₓ value associated with the sustained release oral dosage forms of the present invention is associated with a decreased mortality rate as opposed to higher Cₘₐₓ values of other metoprolol tartrate sustained release oral dosage forms. Most preferably the sustained release oral dosage forms of the present invention provide for smooth blood concentration of metoprolol and an adequate and even effect during the dosage interval.

By “sustained release” it is meant for purposes of the present invention that the therapeutically active medicament is released from the formulation at a controlled rate such that therapeutically beneficial blood levels (but below toxic levels) of the medicament are maintained over an extended period of time, e.g., providing a 24 hour therapeutic effect.

The term “environmental fluid” is meant for purposes of the present invention to encompass, e.g., an aqueous solution, such as that used for in-vitro dissolution testing, or gastrointestinal fluid.

The term “Cₘₐₓ” is meant for purposes of the present invention to mean the maximum plasma concentration of a medicament achieved after single dose administration of a dosage form in accordance with the present invention. The term “Cₘₐₓ at steady state” is meant for purposes of the present invention to mean the maximum plasma concentration of a medicament achieved after state administration of a dosage form in accordance with the present invention.

The term “human subject” for purposes of the present invention is a metoprolol naive healthy human volunteer.

The term “human patient” for purposes of the present invention is a human in need of treatment with metoprolol tartrate therapy.

The term “Tₘₐₓ” is meant for purposes of the present invention to mean the elapsed time from administration of a dosage form to the time the Cₘₐₓ of the medicament is achieved.

The term “mean” for purposes of the present invention, when used to define a pharmacokinetic value (e.g., Tₘₐₓ) represents the arithmetic mean value measured across a patient population.

The term “dose proportional” for purposes of the present invention is meant to encompass both “dose-proportional” and “pseudo-dose proportional” Dose-Proportional means that all active and inactive ingredients are in exactly the same proportion between different strengths (e.g., a tablet of 50-mg strength has all the inactive ingredients, exactly half that of a tablet of 100-mg strength, and twice that of a tablet of 25-mg strength). Pseudo-Dose Proportional means that either 1) the portion of the reduced active ingredient amount in the lower strength dosage form is replaced by an inert diluent such that the total tablet weight is same and the ratios of the inactive ingredients to total tablet weight except the inert diluent is the same or 2) the portion of the reduced active ingredient amount in the lower strength dosage form is not replaced by an inert diluent such that the total tablet weight is reduced equal to the lesser active ingredient and the ratios of the inactive ingredients to total tablet weight are the not the same.

The term USP apparatus type III used herein is described e.g., in the United States Pharmacopeia XXV (2002).

### BRIEF DESCRIPTION OF THE DRAWINGS

FIG. 1 is a graphical representation of the Metoprolol Beta₁-Blockade (Exercise Heart Rate % Change from Baseline (E) versus time) for the clinical study of Example 28.

FIG. 2 is a graphical representation of the steady-state average plasma concentrations (concentration versus time) for the clinical study of Example 28.

### DETAILED DESCRIPTION

Metoprolol is a beta₁-selective (cardioselective) adrenoceptor blocking agent. It reduces oxygen demand of the heart, slowing the heart rate and reducing cardiac output at rest and upon exercise; reduces systolic blood pressure; and can also be used in the treatment of migraine or cluster headache, among other things. The present invention is directed in part to sustained release oral dosage forms comprising the tartrate salt of metoprolol and a sustained release excipient; such that the sustained release oral dosage forms provide certain preferred pharmacokinetic parameters.

In preferred embodiments of the present invention, the sustained release excipient is incorporated into a matrix with the metoprolol tartrate which matrix provides for the sustained release of the metoprolol tartrate.

A non-limiting list of suitable sustained-release materials which may be included in a sustained-release excipient according to the invention include hydrophilic and/or hydrophobic materials, such as gums, cellulose ethers, acrylic resins, protein derived materials, waxes, shellac, and oils such as hydrogenated castor oil and hydrogenated vegetable oil. Certain sustained-release polymers include alkylcelluloses such as ethylcellulose, acrylic and methacrylic acid polymers and copolymers; and cellulose ethers, especially hydroxyalkylcelluloses (especially hydroxypropylmethylcellulose) and carboxyalkylcelluloses. Examples of acrylic and methacrylic acid polymers and copolymers include methyl methacrylate, methyl methacrylate copolymers, ethoxyethyl methacrylates, ethyl acrylate, trimethyl ammonioethyl methacrylate, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid)(anhydride), polymethacrylate, polyacrylamide, poly(methacrylic acid anhydride), and glycidyl methacrylate copolymers. Preferred waxes include for example natural and synthetic waxes, fatty acids, fatty alcohols, and mixtures of the same (e.g., beeswax, camauba wax, stearic acid and stearyl alcohol). Examples of gums include, for example and without limitation, heteropolysaccharides such as xanthan gum(s), homopolysaccharides such as locust bean gum, galactans, mannans, vegetable gums such as alginates, gum karaya, pectin, agar, tragacanth, accacia, carrageenan, tragacanth, chitosan, agar, alginic acid, other polysaccharide gums (e.g. hydrocolloids), mixtures of any of the foregoing, and the like. Certain embodiments utilize mixtures of any of the foregoing sustained release materials in the matrix. However, any pharmaceutically acceptable hydrophobic or hydrophilic sustained-release material which is capable of imparting sustained-release of the active agent may be used in accordance with the present invention.

In certain preferred embodiments of the present invention, the sustained release excipient comprises a gelling agent. Preferably the gelling agent in the sustained release excipient is in an amount of from about 10% to about 60% by weight of the final formulation. In certain especially preferred embodiments, the sustained release excipient comprises a gelling agent of a heteropolysaccharide such as xanthan gum, a homopolysaccharide such as locust bean gum, or a mixture of one or more hetero- and one or more homopolysaccharide(s). Heterodisperse excipients, previously disclosed in our U.S. Pat. Nos. 4,994,276, 5,128,143, and 5,135,757, may be utilized in the sustained release excipient of the present invention. For example, in certain embodiments of the present invention, the sustained release excipient comprises a gelling agent of both hetero- and homo-polysaccharides which exhibit synergism, e.g., the combination of two or more polysaccharide gums producing a higher viscosity and faster hydration than that which would be expected by either of the gums alone, the resultant gel being faster-forming and more rigid.

The term “heteropolysaccharide” as used in the present invention is defined as a water-soluble polysaccharide containing two or more kinds of sugar units, the heteropolysaccharide having a branched or helical configuration, and having excellent water-wicking properties and immense thickening properties.

An especially preferred heteropolysaccharide is xanthan gum, which is a high molecular weight (>10⁶) heteropolysaccharide. Other preferred heteropolysaccharides include derivatives of xanthan gum, such as deacylated xanthan gum, the carboxymethyl ether, and the propylene glycol ester.

The homopolysaccharide materials used in the present invention that are capable of cross-linking with the heteropolysaccharide include the galactomannans, i.e., polysaccharides that are composed solely of mannose and galactose. A possible mechanism for the interaction between the galactomannan and the heteropolysaccharide involves the interaction between the helical regions of the heteropolysaccharide and the unsubstituted mannose regions of the galactomannan. Galactomannans that have higher proportions of unsubstituted mannose regions have been found to achieve more interaction with the heteropolysaccharide. Hence, locust bean gum, which has a higher ratio of mannose to galactose, is especially preferred as compared to other galactomannans, such as guar and hydroxypropyl guar.

The combination of xanthan gum with locust bean gum is an especially preferred gum combination for use in the sustained release excipient of the present invention.

In certain preferred embodiments of the present invention, the controlled release properties of the final product are optimized when the ratio of heteropolysaccharide gum to homopolysaccharide gum is from about 3:1 to about 1:3, and most preferably about 1:1. However, the sustained release excipient of the invention may comprise from about 1% to about 99% by weight heteropolysaccharide gum and from about 99% to about 1% by weight homopolysaccharide gum. Preferably the heteropolysaccharide gum is in an amount of from about 5% to about 30% by weight of the final formulation and preferably the homopolysaccharide gum is in an amount of from about 5% to about 30% by weight of the final formulation.

The combination of any homopolysaccharide gums know to produce a synergistic effect when exposed to aqueous solutions may be used in accordance with the present invention. It is also possible that the type of synergism which is present with regard to the gum combination of the present invention could also occur between two homogeneous or two heteropolysaccharides. Other acceptable gelling agents which may be used in the present invention include those gelling agents well-known in the art. Examples include vegetable gums such as alginates, carrageenan, pectin, guar gum, modified starch, hydroxypropylmethylcellulose, methylcellulose, and other cellulosic materials such as sodium carboxymethylcellulose and hydroxypropyl cellulose. This list is not meant to be exclusive.

Preferably the sustained release excipient of the present invention further comprises an inert diluent. The inert diluent of the sustained release excipient preferably comprises a pharmaceutically acceptable saccharide, including a monosaccharide, a disaccharide, or a polyhydric alcohol, and/or mixtures of any of the foregoing. Examples of suitable inert pharmaceutical diluents include sucrose, dextrose, lactose, mannitol, microcrystalline cellulose, fructose, xylitol, sorbitol, starches, mixtures thereof and the like. However, it is preferred that a soluble pharmaceutical filler such as lactose, dextrose, mannitol, sucrose, or mixtures thereof be used. In certain embodiments, the inert diluent used in the sustained release excipient is in an amount of from about 20 to about 60% by weight of the sustained release excipient. In certain preferred embodiments, the inert diluent used in the sustained release excipient is in an amount of from about 5% to about 40% by weight of the final formulation. The inert diluent or filler may alternatively comprise a pre-manufactured direct compression diluent as set forth below.

In certain especially preferred embodiments the diluent or filler is mannitol. Mannitol may be used in order to increase the stability of the dosage form by decreasing the susceptibility of the drug (metoprolol tartrate) to a a Maillard-type (degradation) reaction. For example, in certain embodiments, the present invention is directed to a method of preventing or reducing a mallaird-type reaction in a metoprolol tartrate sustained release oral dosage form comprising preparing said sustained release oral dosage form by combining a therapeutically effective amount of metoprolol tartrate with a sustained release excipient that provides for the sustained release of said metoprolol tartrate, and including in said dosage form an effective amount of mannitol to prevent or reduce the degradation of said metoprolol tartrate. Preferably, the mannitol is included in the sustained release excipient prior to combining said excipient with said metoprolol tartrate. Alternatively, or additionally, the mannitol is incorporated into said dosage when said metoprolol tartrate and said sustained release excipient before, during, or after the combination of the metoprolol tartrate with the sustained release excipient.

In certain embodiments, the ingredients of the sustained release excipient can be pre-manufactured. However, in other embodiments the active drug can be added to the sustained release excipient ingredients and that mixture wet granulated or spray granulated to form a granulation.

In certain embodiments, it is possible to dry mix the ingredients of the sustained release excipient without utilizing a wet granulation step. This procedure may be utilized, for example, where a wet granulation is to be accomplished when the active ingredient is directly added to the ingredients of the sustained release excipient. On the other hand, this procedure may also be used where no wet granulation step whatsoever is contemplated. If the mixture is to be manufactured without a wet granulation step, and the final mixture is to be tableted, it is preferred that all or part of the inert diluent comprise a pre-manufactured direct compression diluent. Such direct compression diluents are widely used in the pharmaceutical arts, and may be obtained from a wide variety of commercial sources. Examples of such pre-manufactured direct compression excipients include Emcocel® (microcrystalline cellulose, N.F.), Emdex® (dextrates, N.F.), and Tab-Fine® (a number of direct-compression sugars including sucrose, fructose and dextrose), all of which are commercially available from Penwest Pharmaceuticals Co., Patterson, N.Y.). Other direct compression diluents include Anhydrous lactose (Lactose N.F., anhydrous direct tableting) from Sheffield Chemical, Union, N.J. 07083; Elcems® G-250 (powdered cellulose), N.F.) from Degussa, D-600 Frankfurt (Main) Germany; Fast-Flo Lactose® (Lactose, N.F., spray dried) from Foremost Whey Products, Banaboo, Wis. 53913; Maltrin® (Agglomerated maltodextrin) from Grain Processing Corp., Muscatine, Iowa 52761; Neosorb 60® (Sorbitol, N.F., direct-compression from Roquet Corp., 645 5th Ave., New York, N.Y. 10022; Nu-Tab® (Compressible sugar, N.F.) from Ingredient Technology, Inc., Pennsauken, N.J. 08110; Polyplasdone XL® (Crospovidone, N.F., cross-linked polyvinylpyrrolidone) from GAF Corp., New York, N.Y. 10020; Primojel® (Sodium starch glycolate, N.F., carboxymethyl starch) from Generichem Corp., Little Falls, N.J. 07424; Solka Floc® (Cellulose floc) from Penwest Pharmaceuticals Co., Patterson, N.Y. 10512; Spray-dried lactose® (Lactose N.F., spray dried) from Foremost Whey Products, Baraboo, Wis. 53913 and DMV Corp., Vehgel, Holland; and Sta-Rx 1500® (Starch 1500) (Pregelatinized starch, N.F., compressible) from Colorcon, Inc., West Point, Pa. 19486.

In further embodiments of the present invention, the directly compressible inert diluent which is used in conjunction with the sustained release excipient of the present invention is an augmented microcrystalline cellulose as disclosed in U.S. patent application Ser. No. 08/370,576, filed Jan. 9, 1995, and entitled “PHARMACEUTICAL EXCIPIENT HAVING IMPROVED COMPRESSIBILITY”, by J. Staniforth, B. Sherwood and E. Hunter, hereby incorporated by reference in its entirety. The augmented microcrystalline cellulose described therein is commercially available under the tradename “Prosolv” from Penwest Pharmaceuticals Co.

The sustained release excipients prepared in accordance with the present invention may be prepared according to any agglomeration technique to yield an acceptable sustained release excipient product. In wet granulation techniques, the desired amounts of the heteropolysaccharide gum, the homopolysaccharide gum, and the inert diluent are mixed together and thereafter a moistening agent such as water, propylene glycol, glycerol, alcohol or the like is added to prepare a moistened mass. Next, the moistened mass is dried. The dried mass is then milled with conventional equipment into granules. Thereafter, the excipient product is ready to use. The granulate form has certain advantages including the fact that it can be optimized for flow and compressibility; it can be tableted, formulated in a capsule, extruded and spheronized with an active medicament to form pellets, etc.

In certain embodiments of the invention where the sustained release excipient comprises a heteropolysaccharide, a homopolysaccharide, or both, a release-modifying agent may also be incorporated in the formulations (e.g., in the sustatained release excipient) of the present invention. Such release-modifying agents and pre-manufactured excipients disclosed in our U.S. Pat. Nos. 5,455,046; 5,512,297; 5,554,387; 5,667,801; 5,846,563; 5,773,025; 6,048,548; 5,662,933; 5,958,456; 5,472,711; 5,670,168; and 6,039,980 may be utilized in the present invention.

Thus, for example, the release-modifying agent may comprise an ionizable gel-strength enhancing agent. The ionizable gel strength-enhancing agent that is optionally used in conjunction with the present invention may be monovalent or multivalent metal cations. The preferred salts are the inorganic salts, including various alkali metal and/or alkaline earth metal sulfates, chlorides, borates, bromides, citrates, acetates, lactates, etc. Specific examples of suitable ionizable gel strength enhancing agent include calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride, and mixtures thereof. Multivalent metal cations may also be utilized. However, the preferred ionizable gel strength-enhancing agents are bivalent. Particularly preferred salts are calcium sulfate and sodium chloride. In a particular preferred embodiment, the ionizable gel strength-enhancing agent is calcium sulfate dihydrate. The ionizable gel strength enhancing agents of the present invention are added in an amount effective to obtain a desirable increased gel strength due to the cross-linking of the gelling agent (e.g., the heteropolysaccharide and homopolysaccharide gums). In certain embodiments, the ionizable gel strength-enhancing agent is included in the sustained release excipient of the present invention in an amount from about 1 to about 20% by weight of the sustained release excipient, and in an amount 0.5% to about 16% by weight of the final dosage form.

In certain embodiments, the release-modifying agent may comprise a surfactant. Surfactants that may be used in the present invention generally include pharmaceutically acceptable anionic surfactants, cationic surfactants, amphoteric (amphipathic/amphophilic) surfactants, and non-ionic surfactants. Suitable pharmaceutically acceptable anionic surfactants include, for example, monovalent alkyl carboxylates, acyl lactylates, alkyl ether carboxylates, N-acyl sarcosinates, polyvalent alkyl carbonates, N-acyl glutamates, fatty acid-polypeptide condensates, sulfuric acid esters, alkyl sulfates (including sodium lauryl sulfate (SLS)), ethoxylated alkyl sulfates, ester linked sulfonates (including docusate sodium or dioctyl sodium succinate (DSS)), alpha olefin sulfonates, and phosphated ethoxylated alcohols.

Suitable pharmaceutically acceptable cationic surfactants include, for example, monoalkyl quaternary ammonium salts, dialkyl quaternary ammonium compounds, amidoamines, and aminimides.

Suitable pharmaceutically acceptable amphoteric (amphipathic/amphophilic) surfactants, include, for example, N-substituted alkyl amides, N-alkyl betaines, sulfobetaines, and N-alkyl β-aminoproprionates.

Other suitable surfactants for use in conjunction with the present invention include polyethyleneglycols as esters or ethers. Examples include polyethoxylated castor oil, polyethoxylated hydrogenated castor oil, or polyethoxylated fatty acid from castor oil or polyethoxylated fatty acid from hydrogenated castor oil. Commercially available surfactants that can be used are known under trade names Cremophor, Myrj, Polyoxyl 40 stearate, Emerest 2675, Lipal 395 and PEG 3350.

Other release-modifying pharmaceutically acceptable agents that may be added in appropriate quantities for their particular ability to modify dissolution rates include, for example: stearic acid, metallic stearates, stearyl alcohol, hydrogenated cotton seed oil, sodium chloride and certain disintegrants.

The quantity of such release-modifying agent employed depends on the release characteristics required and the nature of the agent. For the sustained release formulation according to the invention, the level of release-modifying agents used may be from about 0.1 to about 25%, preferably from about 0.5 to about 20% by weight of the total composition.

In certain other embodiments of the invention, the sustained release excipient includes a pH-modifying agent. The pH-modifying agent may be present in the sustained release excipient from about 1% to about 10% by weight of the final dosage form. In preferred embodiments, the pH-modifying agent is an organic acid such as citric acid, succinic acid, fumaric acid, malic acid, maleic acid, glutaric acid or lactic acid.

In certain embodiments of the present invention a hydrophobic material is added to the formulation. This may be accomplished by granulating the sustained release excipient with a solution or dispersion of hydrophobic material prior to the incorporation of the medicament. The hydrophobic material may be selected from ethylcellulose, acrylic and/or methacrylic acid polymers or copolymers, hydrogenated vegetable oils, zein, as well as other pharmaceutically acceptable hydrophobic materials known to those skilled in the art. Other hydrophobic cellulosic materials such as other alkyl celluloses may also be used. The amount of hydrophobic material incorporated into the sustained release excipient is that which is effective to slow the hydration of the gums without disrupting the hydrophilic matrix formed upon exposure to an environmental fluid. In certain preferred embodiments of the present invention, the hydrophobic material may be included in the sustained release excipient in an amount from about 1% to about 20% by weight of the sustained release excipient. More preferably, the hydrophobic material may be included in the sustained release excipient in an amount from about 1% to about 10%, and most preferably from about 1% to about 5%, by weight of the final formulation. The hydrophobic material may be dissolved in an organic solvent or dispersed in an aqueous solution for incorporation into the formulation.

Preferably, the sustained release excipients of the invention have uniform packing characteristics over a range of different particle size distributions and are capable of processing into tablets using either direct compression, following addition of drug and lubricant powder, conventional wet granulation, or spray granulation techniques.

In certain embodiments, the properties and characteristics of a specific excipient system prepared according to the present invention is dependent in part on the individual characteristics of the homo- and heteropolysaccharide constituents, in terms of polymer solubility, glass transition temperatures etc., as well as on the synergism both between different homo- and heteropolysaccharides and between the homo and heteropolysaccharides and the inert saccharide constituent(s) in modifying dissolution fluid-excipient interactions.

The oral dosage form of the present invention may be prepared as granules, spheroids, matrix multiparticulates, etc. which comprise metoprolol tartrate in a sustained release matrix, which may be compressed into a tablet or encapsulated.

In certain embodiments, the complete mixture is in an amount sufficient to make a uniform batch of tablets and is subjected to tableting in a conventional production scale tableting machine at normal compression pressure, i.e. about 2000-1600 lbs/sq in. However, the mixture should not be compressed to such a degree that there is subsequent difficulty in achieving hydration when exposed to gastric fluid. The average tablet weight may be from about 100 mg to 950 mg.

In certain preferred embodiments of the present invention, the granules, spheroids, matrix multiparticulates, or tableted formulation are coated with a coating layer which may be comprised of a polymer, mixture of polymers, synthetic and/or naturally occurring, that are freely permeable, slightly permeable, water soluble, water insoluble, and polymers whose permeability and/or solubility is affected by pH.

Preferably the coating comprises a hydrophobic material such as those described above. For example, the hydrophobic material may be a hydrophobic polymer, acrylic and/or methacrylic acid polymers or copolymers, hydrogenated vegetable oils, zein, mixtures thereof, as well as other pharmaceutically acceptable hydrophobic materials known to those skilled in the art. Hydrophobic cellulosic materials such as alkyl celluloses may also be used. In certain embodiments the hydrophobic material in the coating is in an amount of from about 2% to about 15% by weight of the final formulation, preferably from about 2% to about 10% by weight of the final formulation. An especially preferred hydrophobic material is ethylcellulose. Ethylcellulose is commercially available as Aquacoat® (aqueous dispersion of ethylcellulose available from FMC) and Surelease® (aqueous dispersion of ethylcellulose available form Colorcon). In certain preferred embodiments, the ethylcellulose (e.g., aqueous dispersion of ethylcellulose) is mixed with a hydrophilic coating material such a hydroxypropylmethylcellulose (commercially available as Opadry® commercially available from Colorcon, West Point, Pa.) prior to coating the final dosage form.

In other preferred embodiments of the present invention, the hydrophobic material is a pharmaceutically acceptable acrylic polymer, including but not limited to acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), glycidyl methacrylate copolymers, and mixtures thereof.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In order to obtain a desirable dissolution profile, it may be necessary to incorporate two or more ammonio methacrylate copolymers having differing physical properties, such as different molar ratios of the quaternary ammonium groups to the neutral (meth)acrylic esters.

Certain methacrylic acid ester-type polymers are useful for preparing pH-dependent coatings which may be used in accordance with the present invention. For example, there are a family of copolymers synthesized from diethylaminoethyl methacrylate and other neutral methacrylic esters, also known as methacrylic acid copolymer or polymeric methacrylates, commercially available as Eudragit® from Rohm Pharma.

In certain embodiments, a combination of any of the aforementioned hydrophobic materials may be used.

In embodiments of the present invention where the coating comprises an aqueous dispersion of a hydrophobic material, the inclusion of an effective amount of a plasticizer in the aqueous dispersion of hydrophobic material will further improve the physical properties of the sustained release coating.

Examples of suitable plasticizers for ethylcellulose include water insoluble plasticizers such as dibutyl sebacate, diethyl phthalate, triethyl citrate, tributyl citrate, and triacetin, although it is possible that other water-insoluble plasticizers (such as acetylated monoglycerides, phthalate esters, castor oil, etc.) may be used. Triethyl citrate is an especially preferred plasticizer for the aqueous dispersions of ethyl cellulose of the present invention.

Examples of suitable plasticizers for the acrylic polymers of the present invention include, but are not limited to citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit® RL/RS lacquer solutions include polyethylene glycols, propylene glycol, diethyl phthalate, castor oil, and triacetin. Triethyl citrate is also a preferred plasticizer for the acrylic polymers of the present invention.

Such suitable polymers for inclusion into the coating layer preferably slow the release profile of the dosage form.

In other embodiments of the present invention, the coating layer may comprise an enteric coating material in addition to or instead of the hydrophobic polymer coating. Examples of suitable enteric polymers include cellulose acetate phthalate, hydroxypropylmethylcellulose phthalate, polyvinylacetate phthalate, methacrylic acid copolymer, shellac, hydroxypropylmethylcellulose succinate, cellulose acetate trimellitate, and mixtures of any of the foregoing. An example of a suitable commercially available enteric material is available under the trade name Eudragit™ L30D55.

In further embodiments, the dosage form may be coated with a hydrophilic coating in addition to or instead of the above-mentioned coatings. An example of a suitable material which may be used for such a hydrophilic coating is hydroxypropylmethylcellulose (e.g., Opadry® as described above).

The coating layer may be applied in any pharmaceutically acceptable manner known to those skilled in the art. For example, in one embodiment, the coating is applied via a fluidized bed or in a coating pan. For example, the coated tablets may be dried, e.g., at about 60-70° C. for about 3-4 hours in a coating pan. The solvent for the hydrophobic polymer or enteric coating may be organic, aqueous, or a mixture of an organic and an aqueous solvent. The organic solvents may be, e.g., isopropyl alcohol, ethanol, and the like, with or without water.

In additional embodiments of the present invention, a support platform is applied to the tablets manufactured in accordance with the present invention. Suitable support platforms are well known to those skilled in the art. An example of suitable support platform is set forth, e.g., in U.S. Pat. No. 4,839,177, hereby incorporated by reference. In that patent, the support platform partially coats the tablet, and consists of a polymeric material insoluble in aqueous liquids. The support platform may, for example, be designed to maintain its impermeability characteristics during the transfer of the therapeutically active medicament. The support platform may be applied to the tablets, e.g., via compression coating onto part of the tablet surface, by spray coating the polymeric materials comprising the support platform onto all or part of the tablet surface, or by immersing the tablets in a solution of the polymeric materials.

The support platform may have a thickness of, e.g., about 2 mm if applied by compression, and about 10μ if applied via spray-coating or immersion-coating.

Generally, in embodiments of the invention wherein a coating comprising a hydrophobic material or enteric coating material is applied to the tablets, the tablets are coated to a weight gain from about 1 to about 20%, and in certain embodiments preferably from about 5% to about 15%, in certain preferred embodiments from about 7% to about 15%, and in a particular preferred embodiment, about 11%. In certain embodiments, the coating comprising the hydrophobic material is in an amount of from about 1% to about 20, preferably from about 2% to about 15% by weight of the final formulation.

Additionally, the compressed tablets may optionally be coated with a color coat that rapidly disintegrates or dissolves in water or the environment of use. The color coat may be a conventional sugar or polymeric film coating which is applied in a coating pan or by conventional spraying techniques. Preferred materials for the color coat are commercially available under the Opadry tradename (e.g., Opadry® II White, Opadry® II Blue). The color coat may be applied directly onto the tablet core, or may be applied after a coating as described above. Generally, the color coat surrounding the core will comprise from about 1 to 5% preferably about 2 to 4% based on the total weight of the tablet.

An effective amount of any generally accepted pharmaceutical lubricant or mixture of lubricants, including the calcium or magnesium soaps may be added to the above-mentioned ingredients of the formulation at the time the medicament is added, or in any event prior to compression into a solid dosage form. Preferably the lubricant is in an amount of from about 0.5% to about 10%, more preferably from about 0.5% to about 5% by weight of the final formulation. An example of a suitable lubricant is magnesium stearate in an amount of about 0.5% to about 3% by weight of the solid dosage form. An especially preferred lubricant is sodium stearyl fumarate, NF, commercially available under the trade name Pruv® from the Edward Mendell Co., Inc. Another preferred lubricant is talc.

An effective amount of any generally acceptable pharmaceutical glidant or mixture of glidants may also be added to the above-mentioned ingredients of the formulation at the time the medicament is added, or in any event prior to compression into a solid dosage form, including colloidal silicon dioxide, talc, silicon dioxide, sodium aluminosilicate, calcium silicate, powdered cellulose, microcrystalline cellulose, corn starch, sodium benzoate, calcium carbonate, magnesium carbonate, metallic stearates, calcium stearate, magnesium stearate, zinc stearate, stearowet C, starch; starch 1500, magnesium lauryl sulfate, and magnesium oxide. In certain embodiments, a glidant may also be added to the material to be coated prior to application. Preferably the glidant is in an amount of from about 0.5% to about 10%, preferably from about 2% to about 8% by weight of the final formulation.

In certain embodiments, defoaming agents, also known as anti foaming agents, are included in the dosage forms of the present invention. The antifoaming agents are substances used to reduce foaming due to mechanical agitation or to gases, nitrogenous materials or other substances which may interfere during processing. Examples include metallic salts such as sodium chloride; C₆ to C₁₂ alcohols such as octanol; sulfonated oils; silicone ethers such as simethicone; organic phosphates and the like. The amount of antifoaming agent in the composition can range from about 0.005 to about 5%, preferably from about 0.01 to about 2%.

In certain embodiments, additional inert diluent may also be incorporated in the sustained release oral dosage form when mixing the sustained release excipient with the metoprolol tartrate. The inert diluent may be the same or different inert diluent that is incorporated into the sustained release excipient. Other pharmaceutically acceptable diluents and excipients that may be used to formulate oral dosage forms of the present invention are described in the Handbook of Pharmaceutical Excipients, American Pharmaceutical Association (1986).

In order to facilitate the preparation of a sustained-release oral dosage form according to the present invention there is provided, in a further aspect of the present invention, a process for the preparation of the sustained-release oral dosage form according to the present invention comprising incorporating metoprolol tartrate in a sustained-release matrix. Incorporation in the matrix may be effected, for example, by:

(a) forming granules comprising a sustained release excipient comprising at least one hydrophobic and/or hydrophilic material as set forth above together with the metoprolol tartrate;

(b) optionally mixing the sustained release excipient and metoprolol tartrate with the additional ingredients described above;

(c) compressing and shaping the granules into tablets; and

(d) optionally overcoating the tablets with one or more of the coatings described above.

The granules may be formed by any of the procedures well-known to those skilled in the art of pharmaceutical formulation.

In certain embodiments, the sustained release excipients of the present invention are prepared via a wet granulation method. However, the sustained release excipients prepared in accordance with the present invention may be prepared according to any agglomeration technique to yield an acceptable excipient product. In wet granulation techniques, for example, the desired amounts of the heterpolysaccharide gum, the homopolysaccharide gum, optional cationic cross-linking agent and the inert diluent are mixed together and thereafter a moistening agent such as water, propylene glycol, glycerol, alcohol or the like is added to prepare a moistened mass. Next, the moistened mass is dried. The dried mass is then milled with conventional equipment to obtain the desired particle size.

Once the sustained release excipient of the present invention has been prepared, it is then possible to blend the same with the metoprolol tartrate, e.g., in a V-blender and compress the blend into a sustained release oral dosage form.

In certain preferred embodiments, the mixture of sustained release excipient and the active ingredient e.g., metoprolol tartrate (and optionally additional diluent and excipients) may be spray granulated with a solution or suspension of e.g., a cellulose derivative such as an alkylcellulose, hydroxyalkylcellulose, hydroxyalkylalkylcellulose, or mixtures thereof. Preferably, the cellulose derivative is an alkylcellulose such as ethylcellulose, methylcellulose, and the like; a hydroxylalkylcellulose such as hydroxyethylcellulose, hydroxypropylcellulose, and the like; a hydroxylalkylalkylcellulose such as hydroxypropylmethylcellulose, hydroxyethylmethylcellulose, and the like; or mixtures thereof. In certain alternate embodiments, the sustained release excipient may be spray granulated with the cellulose derivative prior to incorporation of the active ingredient, e.g., metoprolol tartrate. Preferably the cellulose derivative used in the spray granulation (e.g., hydroxypropylmethylcellulose) is in the final formulation in an amount of from about 1% to about 10%, preferably from about 2 to about 6% by weight of the final formulation. Preferably the inclusion of the cellulose derivative via spray granulation aids the processing (e.g., tableting) of the formulations (e.g., decreases sticking of granulated powders to the tablet press).

Preferably the granules are compressed into tablets. Although tablets are preferred dosage forms of the present invention, the ingredients may also be formulated in a capsule, extruded and spheronized with an active medicament to form pellets, etc.

In certain preferred embodiments, after the granules are compressed into tablets, the tablets are overcoated with one or more of the coatings described above.

The average particle size of the granulated excipient of the present invention ranges from about 50 microns to about 500 microns and preferably from about 150 microns to about 400 microns. The particle size of the granulation is not narrowly critical, the important parameter being that the average particle size of the granules, must permit the formation of pharmaceutically acceptable tablets. The desired tap and bulk densities of the granulation of the present invention are normally between from about 0.2 to about 0.8 g/ml, with an average density of from about 0.3 to about 0.7 g/ml. For best results, the tablets formed from the granulations of the present invention are from about 12 to about 15 kP hardness.

The amount of metoprolol tartrate incorporated into the sustained release oral dosage forms of the present invention is included in an amount of from about 12.5 mg to about 400 mg, preferably from about 25 to about 200 mg. In certain preferred embodiments, the sustained release oral dosage forms of the present invention comprises metoprolol tartrate in an amount of 12.5 mg, 25 mg, 50 mg, 100 mg, or 200 mg.

### DETAILED DESCRIPTION OF PREFERRED EMBODIMENTS

The following examples illustrate various aspects of the present invention. They are not to be construed to limit the claims in any manner whatsoever.

### EXAMPLE 1

In Example 1, a sustained release excipient in accordance with the present invention is prepared having the following formula, listed in Table 1:

TABLE 1

| Component | Amount (%) |
|---|---|
| Xanthan Gum | 20% |
| Locust Bean Gum | 30% |
| Calcium Sulfate Dihydrate | 10% |
| Mannitol, USP | 40% |
| Water | * |

*Removed during processing

The sustained release excipient of Example 1 is prepared using the following process:

1. Granulating:

a. The mannitol, locust bean gum, xanthan gum and calcium sulfate dihydrate are charged into a Fielder Mixer (M400) and dry mixed for 3 minutes with the granulator and impeller set at slow speed (1).

b. With the granulator and impeller set a slow speed (1), purified water (20-40) kg is added to the mixed powders (Target addition time: 2 min. ±15 sec.).

c. Following the addition of the purified water, the material is granulated for 2-5 minutes with the granulator and impeller set at slow speed (1).

d. Following granulation at slow speed (1), the granulation is checked and additional purified water is added, if required. The material is granulated for an additional 0-2 minutes at slow speed (1) following the water addition.

e. The material is granulated for an additional 0.5-3 minutes with the granulator and impeller set at fast speed (11) following slow speed granulation.

2. Drying

The wet granulation is transferred to a Calmic Fluid Bed Dryer (B305) and dried at an inlet air setpoint of 70° C. until a loss on drying (LOD) of 2-4% is obtained.

3. Milling

The dried granulation is transferred via a Vacuum-Ex transfer system (V 110) through a Fitzpatrick Comminuting Machine (M402/403/404) into the Morley blender (M201).

4. Blending

The milled granulation is blended for 5 minutes in the Morley blender (M201) and discharged into fiber drums which have been lined with two (2) polyethylene bags.

### EXAMPLES 2-5

In Examples 2-5, sustained release oral dosage forms were prepared having the formulas listed in the table below:

TABLE 2

| | Example 2 | Example 3 | Example 4 | Example 5 |
|---|---|---|---|---|
| Component | (mg/tab) | (mg/tab) | (mg/tab) | (mg/tab) |
| Sustained Release | 297.0 | 297.0 | 297.0 | 297.0 |
| Excipient of Example 1 | | | | |
| Metoprolol Tartrate | 200 | 100 | 50.0 | 25.0 |
| Mannitol, powdered | — | 100 | — | 25.0 |
| Methocel E-5LV | 22.2 | 22.2 | 15.5 | 15.5 |
| (Hydroxypropylmethyl- | | | | |
| cellulose) | | | | |
| Alpha-Fil 500 (talc) | 27.5 | 27.5 | 19.7 | 19.7 |
| Pruv (Sodium stearyl | 8.3 | 8.3 | 5.8 | 5.8 |
| fumarate) | | | | |
| TOTAL | 555.0 | 555.0 | 388.0 | 388.0 |

Preparation of Unit Dosage Forms:

1. Dissolve the hydroxypropylmethylcellulose in the requisite amount of water to provide for a spray granulating solution with 10% solids.

2. Screen the solution through a #20 screen.

3. Charge an Nitro-Fielder MP-1 granulator with the requisite amounts of sustained release excipient, metoprolol tartrate, hydroxypropylmethylcellulose, and mannitol (if indicated).

4. Preheat the materials for 10 minutes. (approximate values)

| | | | |
|---|---|---|---|
| a. | Inlet Temp.: | 70° | C. |
| b. | Bed Temp.: | 40-45° | C. |
| c. | Air Flow: 50-60 | CMH | |
| d. | Blowback pressure: | 5.0 | bar |
| e. | Atm air: | 15% | (1.0 bar) |

5. Start spray:

| | |
|---|---|
| a. Inlet Temp.: | 70° C. |
| b. Bed Temp.: | 30-40° C. |
| c. Air Flow: | 60-80 CMH |
| d. Blowback pressure: | 5.0 bar |
| e. Atm air: | 27% (1.8 bar) |
| f. Pump speed: | TBD |

6. After the desired amount has been sprayed, dry the granulation to an LOD of less than 4%.

| | |
|---|---|
| a. Inlet Temp.: | 70° C. |
| b. Bed Temp.: | 35-50° C. |
| C. Air Flow: | 50-60 CMH |
| d. Blowback pressure: | 5.0 bar |

7. Hand screen the granulation through a # 25 sieve.

8. Blend the granulation in a 16qt Patterson Kelly-V-blender with the requisite amount of Alpha-Fil 500 for 10 minutes and then the requisite amount of Pruv for 5 minutes.

9. Compress the material into tablets using a CadMach DC16 press.

### EXAMPLE 6

The tablets prepared in accordance with Examples 2-5 were dissolution tested.

Tablets having the formulation of Example 2 were dissolution tested using USP dissolution Apparatus Type III in 250 ml of 0.1 M pH 7.5, at 15 dpm, and gave the results listed in Table 3 below:

TABLE 3

| Time | % dissolved | Std. Dev. |
|---|---|---|
| 0 | 0.0 | 0.0 |
| 1 | 29.2 | 0.7 |
| 3 | 54.0 | 1.4 |
| 6 | 80.6 | 1.5 |
| 8 | 93.3 | 1.5 |
| 16 | 106.1 | 1.4 |
| 24 | 106.6 | 1.4 |
| Remnant | 0.0 | 0.0 |
| Total | 106.6 | 1.4 |

Tablets having the formulation of Example 3 were dissolution tested using USP dissolution Apparatus Type III in 250 ml of 0.1 M pH 7.5, at 15 dpm, and gave the results listed in Table 4 below:

TABLE 4

| Time | % dissolved | Std. Dev. |
|---|---|---|
| 0 | 0.0 | 0.0 |
| 1 | 26.4 | 0.9 |
| 3 | 49.0 | 1.3 |
| 6 | 74.1 | 2.1 |
| 8 | 84.8 | 2.4 |
| 16 | 96.4 | 2.8 |
| 24 | 97.3 | 2.7 |
| Remnant | 0.0 | 0.0 |
| Total | 97.3 | 2.7 |

Tablets having the formulation of Example 4 were dissolution tested using USP dissolution Apparatus Type III in 250 ml of 0.1 M pH 7.5, at 15 dpm, and gave the results listed in Table 5 below:

TABLE 5

| Time | % dissolved | Std. Dev. |
|---|---|---|
| 0 | 0.0 | 0.0 |
| 1 | 26.6 | 0.6 |
| 3 | 49.7 | 0.8 |
| 6 | 73.3 | 1.6 |
| 8 | 84.6 | 2.1 |
| 16 | 101.0 | 2.5 |
| 24 | 102.4 | 2.7 |
| Remnant | 0.0 | 0.0 |
| Total | 102.4 | 2.7 |

Tablets having the formulation of Example 5 were dissolution tested using USP dissolution Apparatus Type III in 250 ml of 0.1 M pH 7.5, at 15 dpm, and gave the results listed in Table 6 below:

TABLE 6

| Time | Mean % dissolved | Std. Dev. |
|---|---|---|
| 0 | 0.0 | 0.0 |
| 1 | 25.8 | 0.9 |
| 3 | 47.8 | 1.9 |
| 6 | 70.8 | 2.6 |
| 8 | 82.7 | 2.9 |
| 16 | 100.4 | 2.6 |
| 24 | 101.4 | 2.6 |
| Remnant | 0.2 | 0.1 |
| Total | 101.5 | 2.5 |

### EXAMPLES 7-10

In Examples 7-10, the sustained release oral dosage forms prepared in accordance with Examples 2-5 were coated with a sustained release coating and a color coating having the formulas listed in the table below:

TABLE 7

| | Example 7 | Example 8 | Example 9 | Example 10 |
|---|---|---|---|---|
| Component | (mg/tab) | (mg/tab) | (mg/tab) | (mg/tab) |
| Example 2 tablet | 555.0 | — | — | — |
| Example 3 tablet | — | 555.0 | — | — |
| Example 4 tablet | — | — | 388.0 | — |
| Example 5 tablet | — | — | — | 388.0 |
| Surelease E-7-7050 | 48.8 | 48.8 | 34.2 | 34.2 |
| Opadry II Clear | 12.2 | 12.2 | 8.5 | 8.5 |
| Opadry II White or | 18.5 | 18.5 | 12.9 | 12.9 |
| Blue | | | | |
| TOTAL | 634.5 | 634.5 | 443.6 | 443.6 |

Process:

1. Using a vector LDCS coating unit, coat the tablets with a Surelease/Opadry II clear 80/20 ratio of a 15% dispersion to a weight gain of 11% using the following settings:

| | |
|---|---|
| a. Inlet air temp.: | 55-75° C. |
| b. Exhaust air temp.: | 25-40° C. |
| c. Air volume: | 20-40 cfm |
| d. Pan rotation: | 5.0 rpm |
| e. Atm air: | 15% (1.0 bar) |

2. Dry the tablets for 15-20 minutes (bed temp 40 C) and allow the tablets to cool to approximately room temperature before discharging.

3. Coat the tablets with a suspension of 15% of Opadry II, white (or blue) and water, to a weight gain of approximately 3%.

### EXAMPLE 11

The tablets prepared in accordance with Examples 7-10 were dissolution tested.

Tablets having the formulation of Example 7 were dissolution tested using USP dissolution Apparatus Type III in 250 ml of 0.1 M pH 7.5, at 15 dpm, and gave the results listed in Table 8 below:

TABLE 8

| Time | % dissolved | Std. Dev. |
|---|---|---|
| 0 | 0.0 | 0.0 |
| 1 | 0.5 | 0.5 |
| 3 | 14.4 | 2.7 |
| 6 | 36.7 | 2.8 |
| 8 | 50.0 | 3.0 |
| 16 | 87.6 | 2.9 |
| 24 | 103.7 | 2.2 |
| Remnant | 2.4 | 1.1 |
| Total | 106.1 | 2.6 |

Tablets having the formulation of Example 8 were dissolution tested using USP dissolution Apparatus Type III in 250 ml of 0.1 M pH 7.5, at 15 dpm, and gave the results listed in Table 9 below:

TABLE 9

| Time | % dissolved | Std. Dev. |
|---|---|---|
| 0 | 0.0 | 0.0 |
| 1 | 0.0 | 0.0 |
| 3 | 10.3 | 1.0 |
| 6 | 31.4 | 0.8 |
| 8 | 44.1 | 0.9 |
| 16 | 81.3 | 1.9 |
| 24 | 96.0 | 3.0 |
| Remnant | 3.8 | 0.6 |
| Total | 99.8 | 3.1 |

Tablets having the formulation of Example 9 were dissolution tested using USP dissolution Apparatus Type III in 250 ml of 0.1 M pH 7.5, at 15 dpm, and gave the results listed in Table 10 below:

TABLE 10

| Time | % dissolved | Std. Dev. |
|---|---|---|
| 0 | 0.0 | 0.0 |
| 1 | 1.7 | 0.5 |
| 3 | 17.9 | 1.2 |
| 6 | 40.6 | 1.6 |
| 8 | 54.7 | 2.7 |
| 16 | 88.1 | 4.2 |
| 24 | 100.7 | 3.2 |
| Remnant | 1.8 | 0.6 |
| Total | 102.6 | 2.7 |

Tablets having the formulation of Example 10 were dissolution tested using USP dissolution Apparatus Type III in 250 ml of 0.1 M pH 7.5, at 15 dpm, and gave the results listed in Table 11 below:

TABLE 11

| Time | % dissolved | Std. Dev. |
|---|---|---|
| 0 | 0.0 | 0.0 |
| 1 | 1.0 | 0.6 |
| 3 | 15.9 | 1.5 |
| 6 | 36.7 | 1.7 |
| 8 | 49.0 | 2.5 |
| 16 | 81.6 | 5.0 |
| 24 | 98.3 | 4.6 |
| Remnant | 3.2 | 1.2 |
| Total | 101.4 | 4.0 |

### EXAMPLE 12

In Example 12, a sustained release excipient in accordance with the present invention is prepared having the formulation below:

TABLE 12

| Component | Amount % |
|---|---|
| 1. Xanthan Gum | 25 |
| 2. Locust Bean Gum | 25 |
| 3. Dextrose | 35 |
| 4. Calcium Sulfate Dihydrate | 10 |
| 5. Ethylcellulose | 5 |
| 5. Alcohol, SD3A, anhydrous* | 20 |
| 6. Water* | q.s. |

*Removed during processing

Process:

1. The requisite amounts of xanthan gum, locust bean gum, calcium sulfate, and dextrose are dry blended in a high speed mixer/granulator for 3 minutes.

2. A slurry of hydrophobic polymer (ethylcellulose) is prepared by dissolving ethyl cellulose in ethyl alcohol.

3. The slurry is added to the dry blended mixture, and granulated for another 3 minutes.

4. The granulation was then dried in a fluid bed dryer to a LOD (loss on drying) of less than about 10% by weight (e.g., 4-7% LOD).

### EXAMPLES 13-16

In Examples 13-16, sustained release oral dosage forms were prepared having the formulas listed in the table below:

TABLE 13

| | Example 13 | Example 14 | Example 15 | Example 16 |
|---|---|---|---|---|
| Component | (mg/tab) | (mg/tab) | (mg/tab) | (mg/tab) |
| Sustained Release Excipient of | 297.0 | 297.0 | 297.0 | 297.0 |
| Example 12 | | | | |
| Metoprolol Tartrate | 200 | 100 | 50.0 | 25.0 |
| Dextrose | — | 100 | — | 25.0 |
| Methocel E-5LV | 22.2 | 22.2 | 15.5 | 15.5 |
| (Hydroxypropylmethylcellulose) | | | | |
| Alpha-Fil 500 (talc) | 27.5 | 27.5 | 19.7 | 19.7 |
| Pruv (Sodium stearyl fumarate) | 8.3 | 8.3 | 5.8 | 5.8 |
| TOTAL | 555.0 | 555.0 | 388.0 | 388.0 |

Process:

The formulations of Examples 13-16 are prepared in accordance with the procedures set forth in Examples 2-5.

### EXAMPLE 17

The tablets prepared in accordance with Examples 13-16 were dissolution tested.

Tablets having the formulation of Example 13 were dissolution tested using USP dissolution Apparatus Type III in 250 ml of 0.1 M pH 7.5, at 15 dpm, and gave the results listed in Table 14 below:

TABLE 14

| Time | % dissolved | Std. Dev. |
|---|---|---|
| 0 | 0.0 | 0.0 |
| 1 | 26.3 | 0.1 |
| 3 | 49.5 | 0.2 |
| 6 | 73.8 | 1.2 |
| 8 | 85.6 | 1.2 |
| 16 | 100.5 | 1.4 |
| 24 | 102.1 | 1.4 |
| Remnant | 0.1 | 0.1 |
| Total | 102.1 | 1.4 |

Tablets having the formulation of Example 14 were dissolution tested using USP dissolution Apparatus Type III in 250 ml of 0.1 M pH 7.5, at 15 dpm, and gave the results listed in Table 15 below:

TABLE 15

| Time | % dissolved | Std. Dev. |
|---|---|---|
| 0 | 0.0 | 0.0 |
| 1 | 24.7 | 0.4 |
| 3 | 47.1 | 0.3 |
| 6 | 71.1 | 0.8 |
| 8 | 81.7 | 0.7 |
| 16 | 94.5 | 0.7 |
| 24 | 96.1 | 1.0 |
| Remnant | 0.1 | 0.0 |
| Total | 96.2 | 1.0 |

Tablets having the formulation of Example 15 were dissolution tested using USP dissolution Apparatus Type III in 250 ml of 0.1 M pH 7.5, at 15 dpm, and gave the results listed in Table 16 below:

TABLE 16

| Time | % dissolved | Std. Dev. |
|---|---|---|
| 0 | 0.0 | 0.0 |
| 1 | 22.3 | 0.1 |
| 3 | 42.1 | 0.5 |
| 6 | 62.8 | 1.0 |
| 8 | 73.4 | 1.8 |
| 16 | 90.9 | 1.7 |
| 24 | 94.5 | 1.6 |
| Remnant | 0.4 | 0.1 |
| Total | 94.8 | 1.6 |

Tablets having the formulation of Example 16 were dissolution tested using USP dissolution Apparatus Type III in 250 ml of 0.1 M pH 7.5, at 15 dpm, and gave the results listed in Table 17 below:

TABLE 17

| Time | % dissolved | Std. Dev. |
|---|---|---|
| 0 | 0.0 | 0.0 |
| 1 | 24.2 | 0.4 |
| 3 | 46.8 | 1.1 |
| 6 | 70.7 | 2.4 |
| 8 | 82.7 | 2.4 |
| 16 | 102.5 | 2.1 |
| 24 | 106.6 | 1.8 |
| Remnant | 0.0 | 0.0 |
| Total | 106.6 | 1.8 |

### EXAMPLES 18 and 19

In Examples 18 and 19, the sustained release oral dosage forms prepared in accordance with Examples 13 and 16 were coated with a sustained release coating and a color coating having the formulas listed in the table below:

TABLE 18

| | Example 18 | Example 19 |
|---|---|---|
| Component | (mg/tab) | (mg/tab) |
| Example 13 tablet | 555.0 | — |
| Example 16 tablet | — | 388.0 |
| Surelease E-7-7050 | 48.8 | 34.2 |
| Opadry II Clear | 12.2 | 8.5 |
| Opadry II White or Blue | 18.5 | 12.9 |
| TOTAL | 634.5 | 443.6 |

Process:

The formulations of Examples 18 and 19 are prepared in accordance with the procedures set forth in Examples 7-10.

### EXAMPLE 20

The tablets prepared in accordance with Examples 18 and 19 were dissolution tested.

Tablets having the formulation of Example 18 were dissolution tested using USP dissolution Apparatus Type III in 250 ml of 0.1 M pH 7.5, at 15 dpm, and gave the results listed in Table 19 below:

TABLE 19

| Time | % dissolved | Std. Dev. |
|---|---|---|
| 0 | 0.0 | 0.0 |
| 1 | 0.4 | 0.4 |
| 3 | 13.6 | 1.2 |
| 6 | 34.2 | 1.7 |
| 8 | 47.1 | 1.8 |
| 16 | 82.9 | 3.7 |
| 24 | 98.8 | 2.7 |
| Remnant | 2.3 | 1.3 |
| Total | 101.1 | 1.9 |

Tablets having the formulation of Example 19 were dissolution tested using USP dissolution Apparatus Type III in 250 ml of 0.1 M pH 7.5, at 15 dpm, and gave the results listed in Table 20 below:

TABLE 20

| Time | % dissolved | Std. Dev. |
|---|---|---|
| 0 | 0.0 | 0.0 |
| 1 | 2.4 | 0.3 |
| 3 | 17.6 | 1.0 |
| 6 | 44.2 | 1.6 |
| 8 | 58.6 | 1.5 |
| 16 | 91.6 | 1.2 |
| 24 | 101.5 | 1.1 |
| Remnant | 2.5 | 0.3 |
| Total | 104.0 | 1.0 |

### EXAMPLE 21

A biostudy was conducted utilizing the formulations prepared in accordance with Examples 13 and 18. Additional formulations having 7% coating of 80/20 ethylcellulose/opadry and 7% coating of 90/10 ethylcellulose/opadry were also used in the biostudy. The following treatments correspond with the following formulations:

Treatment A: formulations of Example 18

Treatment B: formulations of Example 13 with a 7% coating of 80/20 ethylcellulose/opadry prepared in accordance with the process of Example 18.

Treatment C: formulations of Example 13 with a 7% coating of 90/10 ethylcellulose/opadry prepared in accordance with the process of Example 18.

Treatment D: Lopressor®) 100 mg (immediate release) tablet

The biostudy was conducted under both Fed and Fasting conditions and gave the following results listed in the tables below:

TABLE 21a
Summary Statistics for Dose Normalized Pharmacokinetic Parameters (AUCtlast, AUCinf) by
Treatment under fasting conditions

| Pk Parameter | Statistics | Treatment A | Treatment B | Treatment C | Treatment D |
|---|---|---|---|---|---|
| AUCtlast(ng- | N | 10 | 10 | 10 | 10 |
| h/ML) | AM ± SD | 731.12 ± | 675.56 ± | 844.21 ± | 937.82 ± 489.77 |
| | | 515.61 | 398.20 | 487.51 | |
| | GM | 521.37 | 538.49 | 690.07 | 813.38 |
| | CV % | 70.5% | 58.9% | 57.7% | 52.2% |
| | Range | 100.59- | 213.99- | 268.71- | 384.91-1717.81 |
| | | 1475.84 | 1380.30 | 1543.73 | |
| | GM Ratio¹ | 0.64 | 0.66 | 0.84 | |
| | 90% CI for GM | (0.497, 0.826) | (0.513, 0.855) | (0.658, 1.094) | |
| | Ratio¹ | | | | |
| AUCinf(ng-h/mL) | N | 7 | 9 | 7 | 10 |
| | AM ± SD | 921.21 ± | 740.10 ± | 1021.7 ± | 957.10 ± 486.11 |
| | | 511.74 | 385.05 | 499.40 | |
| | GM | 607.14 | 560.04 | 710.32 | 840.88 |
| | CV % | 55.6% | 52.0% | 48.9% | 50.8% |
| | Range | 265.95- | 258.29- | 276.49- | 416.88-1730.71 |
| | | 1502.71 | 1386.48 | 1562.80 | |
| | GM Ratio¹ | 0.72 | 0.66 | 0.84 | |
| | 90% CI for GM | (0.521, 1.001) | (0.493, 0.9) | (0.606, 1.177) | |
| | Ratio¹ | | | | |
| Cmax(ng/mL) | N | 10 | 10 | 10 | 10 |
| | AM ± SD | 47.19 ± 32.28 | 54.52 ± 30.43 | 51.82 ± 31.62 | 201.78 ± 83.86 |
| | GM | 35.43 | 43.22 | 43.09 | 183.02 |
| | CV % | 68.4% | 55.8% | 61.0% | 41.6% |
| | Range | 11.31-97.41 | 10.66-108.32 | 18.12-104.96 | 89.59-350.37 |
| | GM Ratio¹ | 0.19 | 0.23 | 0.23 | |
| | 90% CI for GM | (0.16, 0.234) | (0.195, 0.285) | (0.195, 0.284) | |
| | Ratio¹ | | | | |

AUCtlast = Area under the concentration-time curve C(t) from time zero to the last measurable sampling time point, t
AUCinf = Area under the concentration-time curve from time zero to infinity
Cmax = Maximum observed post-dose concentration
AM = Arithmetic mean, SD = Standard Deviation, GM = Geometric Mean, CV = Coefficient of Variation
¹Ratio of the test formulation to the reference formulation

TABLE 21b
Descriptive Statistics for Pharmacokinetic Parameters (Tmax, Kel t½)
by Treatment under fasting conditions

| Pk Parameter | Statistics | Treatment A | Treatment B | Treatment C | Treatment D |
|---|---|---|---|---|---|
| Tmax | N | 10 | 10 | 10 | 10 |
| | AM ± SD | 15.40 ± 4.81 | 9.00 ± 3.53 | 15.20 ± 4.54 | 1.65 ± 0.47 |
| | GM | 31.2% | 39.2% | 29.9% | 28.7% |
| | CV % | 16.00 | 8.00 | 16.00 | 1.50 |
| | Range | 6.00-24.00 | 5.00-16.00 | 8.00-24.00 | 1.00-2.50 |
| Kel | N | 7 | 9 | 7 | 10 |
| | AM ± SD | 0.20 ± 0.04 | 0.19 ± 0.02 | 0.19 ± 0.05 | 0.24 ± 0.06 |
| | GM | 18.8% | 10.3% | 24.9% | 24.5% |
| | CV % | 0.19 | 0.20 | 0.21 | 0.25 |
| | Range | 0.16-0.27 | 0.17-0.22 | 0.13-0.25 | 0.15-0.34 |
| t½ | N | 7 | 9 | 7 | 10 |
| | AM ± SD | 3.55 ± 0.63 | 3.62 ± 0.37 | 3.89 ± 1.03 | 3.04 ± 0.83 |
| | GM | 17.6% | 10.3% | 26.6% | 27.3% |
| | CV % | 3.59 | 3.54 | 3.36 | 2.74 |
| | Range | 2.61-4.41 | 3.11-4.19 | 2.81-5.47 | 2.05-4.73 |

Tmax = time to attain Cmax
Kel = apparent elimination rate constant determined by log-linear regression of the terminal log-linear segment of the plasma concentration-time curve
t½ = apparent elimination half-life calculated as 0.693/Kel
SD = Standard Deviation, CV = Coefficient of Variation

TABLE 22a
Summary Statistics for Dose Normalized Pharmacokinetic Parameters (AUCtlast, AUCinf) by
Treatment under Fed conditions

| Pk Parameter | Statistics | Treatment A | Treatment B | Treatment C | Treatment D |
|---|---|---|---|---|---|
| AUCtlast(ng- | N | 11 | 11 | 11 | 11 |
| h/ML) | AM ± SD | 832.42 ± | 930.42 ± | 819.69 ± | 924.56 ± 524.58 |
| | | 548.56 | 714.81 | 648.49 | |
| | GM | 756.81 | 828.68 | 689.77 | 842.18 |
| | CV % | 65.9% | 76.8% | 79.1% | 56.7% |
| | Range | 352.73- | 293.63- | 124.27- | 348.94-1890.62 |
| | | 1965.16 | 2814.52 | 2447.29 | |
| | GM Ratio¹ | 0.89 | 0.98 | 0.81 | |
| | 90% CI for GM | (0.775, 1.042) | (0.849, 1.141) | (0.706, 0.949) | |
| | Ratio¹ | | | | |
| AUCinf(ng-h/mL) | N | 10 | 10 | 10 11 | |
| | AM ± SD | 860.92 ± | 1006.7 ± | 900.08 ± | 941.60 ± 528.91 |
| | | 588.38 | 728.32 | 644.73 | |
| | GM | 756.21 | 861.51 | 753.39 | 863.69 |
| | CV % | 68.3% | 72.3% | 71.6% | 56.2% |
| | Range | 359.60- | 491.59- | 459.83- | 368.27-1912.14 |
| | | 2005.68 | 2853.01 | 2475.85 | |
| | GM Ratio¹ | 0.87 | 0.99 | 0.87 | |
| | 90% CI for GM | (0.769, 0.997) | (0.876, 1.136) | (0.766, 0.994) | |
| | Ratio¹ | | | | |
| Cmax(ng/mL) | N | 11 | 11 | 11 | 11 |
| | AM ± SD | 61.24 ± 34.79 | 87.25 ± 40.67 | 71.47 ± 37.70 | 193.08 ± 92.36 |
| | GM | 57.30 | 82.91 | 62.25 | 177.32 |
| | CV % | 56.8% | 46.6% | 52.7% | 47.8% |
| | Range | 32.32-137.87 | 29.86-150.79 | 11.23-120.65 | 75.66-377.92 |
| | GM Ratio¹ | 0.32 | 0.46 | 0.35 | |
| | 90% CI for GM | (0.254, 0.411) | (0.358, 0.594) | (0.276, 0.446) | |
| | Ratio¹ | | | | |

AUCtlast = Area under the concentration-time curve C(t) from time zero to the last measurable sampling time point, t
AUCinf = Area under the concentration-time curve from time zero to infinity
Cmax = Maximum observed post-dose concentration
AM = Arithmetic mean, SD = Standard Deviation, GM = Geometric Mean, CV = Coefficient of Variation
¹Ratio of the test formulation to the reference formulation

TABLE 22b
Descriptive Statistics for Pharmacokinetic Parameters (Tmax, Kel, t½)
by Treatment under Fed conditions

| Pk Parameter | Statistics | Treatment A | Treatment B | Treatment C | Treatment D |
|---|---|---|---|---|---|
| Tmax | N | 11 | 11 | 11 | 11 |
| | AM ± SD | 14.73 ± 5.53 | 9.82 ± 3.74 | 10.36 ± 4.72 | 2.09 ± 1.34 |
| | GM | 37.6% | 38.1% | 45.5% | 64.0% |
| | CV % | 12.00 | 8.00 | 8.00 | 1.50 |
| | Range | 8.00-24.00 | 6.00-16.00 | 6.00-20.00 | 1.00-5.00 |
| Kel | N | 10 | 10 | 10 | 11 |
| | AM ± SD | 0.19 ± 0.05 | 0.22 ± 0.04 | 0.21 ± 0.05 | 0.22 ± 0.06 |
| | GM | 24.0% | 18.4% | 23.0% | 27.9% |
| | CV % | 0.20 | 0.22 | 0.21 | 0.21 |
| | Range | 0.12-0.28 | 0.12-0.26 | 0.14-0.32 | 0.14-0.38 |
| t½ | N | 10 | 10 | 10 | 11 |
| | AM ± SD | 3.83 ± 0.97 | 3.34 ± 0.86 | 3.41 ± 0.74 | 3.27 ± 0.79 |
| | GM | 25.3% | 25.9% | 21.8% | 24.1% |
| | CV % | 3.56 | 3.12 | 3.24 | 3.30 |
| | Range | 2.48-5.68 | 2.62-5.62 | 2.16-4.80 | 1.81-4.90 |

Tmax = time to attain Cmax
Kel apparent elimination rate constant determined by log-linear regression of the terminal log-linear segment of the plasma concentration-time curve
t½ = apparent elimination half-life calculated as 0.693/Kel
SD = Standard Deviation, CV = Coefficient of Variation

### EXAMPLE 22

A biostudy was conducted utilizing the formulations prepared in accordance with Examples 16 and 19. Additional formulations having 7% coating of 80/20 ethylcellulose/opadry and 7% coating of 90/10 ethylcellulose/opadry were also used in the biostudy. The following treatments correspond with the following formulations:

Treatment A: formulations of Example 19

Treatment B: formulations of Example 16 with a 7% coating of 80/20 ethylcellulose/opadry prepared in accordance with the process of Example 18.

Treatment C: formulations of Example 16 with a 7% coating of 90/10 ethylcellulose/opadry prepared in accordance with the process of Example 18.

Treatment D: one-half of a Lopressor 50 mg (immediate release) tablet

The biostudy was conducted under both Fed and Fasting conditions and gave the following results listed in the tables below:

TABLE 23a
Summary Statistics for Pharmacokinetic Parameters (AUCtlast, AUCinf, Cmax) by
Treatment under Fasting conditions

| Pk Parameter | Statistics | Treatment A | Treatment B | Treatment C | Treatment D |
|---|---|---|---|---|---|
| AUCtlast(ng-h/mL) | N | 12 | 12 | 12 | 12 |
| | AM ± SD | 101.72 ± 86.45 | 92.51 ± 64.13 | 81.21 ± 56.73 | 94.75 ± 77.13 |
| | GM | 77.89 | 74.96 | 62.06 | 72.74 |
| | CV % | 85.0% | 69.3% | 69.9% | 81.4% |
| | Range | 33.98-322.96 | 30.19-191.85 | 9.50-185.24 | 24.97-277.62 |
| | GM Ratio | 1.07 | 1.03 | 0.85 | |
| | 90% CI for GM Ratio¹ | (0923, 1.242) | (0.889, 1.195) | (0.736, 0.989) | |
| AUCinf(ng-h/mL) | N | 8 | 12 | 11 | 12 |
| | AM ± SD | 127.23 ± 99.30 | 93.76 ± 64.52 | 85.02 ± 58.92 | 95.68 ± 77.62 |
| | GM | 84.92 | 76.24 | 62.66 | 73.65 |
| | CV % | 78.0% | 68.8% | 69.3% | 81.1% |
| | Range | 35.71-326.75 | 30.78-193.45 | 10.29-186.39 | 25.70-279.95 |
| | GM Ratio | 1.15 | 1.03 | 0.85 | |
| | 90% CI for GM Ratio¹ | (0.968, 1.374) | (0.894, 1.198) | (0.731, 0.99) | |
| Cmax(ng/mL) | N | 12 | 12 | 12 | 12 |
| | AM ± SD | 5.18 ± 3.88 | 5.44 ± 3.06 | 4.96 ± 3.28 | 17.72 ± 13.59 |
| | GM | 4.18 | 4.75 | 4.14 | 14.16 |
| | CV % | 74.9% | 56.2% | 66.1% | 76.7% |
| | Range | 1.88-13.82 | 2.06-11.46 | 1.65-11.37 | 5.75-50.78 |
| | GM Ratio¹ | 0.29 | 0.33 | 0.29 | |
| | 90% CI for GM Ratio¹ | (0.263, 0.331) | (0.299, 0.377) | (0.26, 0.328) | |

AUCtlast = Area under the concentration-time curve C(t) from time zero to the last measurable sampling time point, t
AUCinf = Area under concentration-time curve from time zero to infinity
Cmax = Maximum observed post-dose concentration
AM = Arithmetic mean, SD = Standard Deviation, GM = Geometric Mean, CV = Coefficient of Variation
¹Ratio of the test formulation to the reference formulation

TABLE 23b
Descriptive Statistics for Pharmacokinetic Parameters (Tmax, Kel, t½)
by Treatment under Fasting conditions

| Pk Parameter | Statistics | Treatment A | Treatment B | Treatment C | Treatment D |
|---|---|---|---|---|---|
| Tmax | N | 12 | 12 | 12 | 12 |
| | AM ± SD | 13.42 ± 6.72 | 8.58 ± 4.48 | 9.42 ± 5.18 | 1.67 ± 0.58 |
| | CV % | 50.1% | 52.2% | 55.0% | 34.6% |
| | Median | 12.00 | 8.00 | 7.00 | 1.50 |
| | Range | 5.00-24.00 | 5.00-20.00 | 5.00-20.00 | 1.00-3.00 |
| Kel | N | 8 | 12 | 11 | 12 |
| | AM ± SD | 0.16 ± 0.04 | 0.16 ± 0.04 | 0.17 ± 0.04 | 0.18 ± 0.04 |
| | CV % | 22.4% | 25.6% | 24.9% | 24.1% |
| | Median | 0.15 | 0.16 | 0.16 | 0.18 |
| | Range | 0.11-0.20 | 0.09-0.23 | 0.09-0.21 | 0.12-0.24 |
| t½ | N | 8 | 12 | II | 12 |
| | AM ± SD | 4.63 ± 1.05 | 4.52 ± 1.33 | 4.46 ± 1.32 | 4.06 ± 1.04 |
| | CV % | 22.6% | 29.3% | 29.6% | 25.6% |
| | Median | 4.65 | 4.25 | 4.24 | 3.89 |
| | Range | 3.48-6.29 | 3.06-7.42 | 3.29-7.49 | 2.48-5.70 |

Tmax = time to attain Cmax
Kel = apparent elimination rate constant determined by log-linear regression of the terminal log-linear segment of the plasma concentration-time curve
t½ = apparent elimination half-life calculated as 0.693/Kel
SD = Standard Deviation, CV = Coefficient of Variation

TABLE 24a
Summary Statistics for Pharmacokinetic Parameters (AUCtlast, AUCinf, Cmax) by
Treatment under Fed Conditions

| Pk Parameter | Statistics | Treatment A | Treatment B | Treatment C | Treatment D |
|---|---|---|---|---|---|
| AUCtlast(ng-h/mL) | N | 11 | 11 | 11 | 11 |
| | AM ± SD | 95.82 ± 51.97 | 109.56 ± 68.33 | 108.79 ± 53.22 | 113.05 ± 52.60 |
| | GM | 82.44 | 99.06 | 102.08 | 106.04 |
| | CV % | 54.2% | 62.4% | 48.9% | 46.5% |
| | Range | 9.74-199.05 | 41.37-267.95 | 45.31-219.97 | 38.62-191.13 |
| | GM Ratio¹ | 0.77 | 0.93 | 0.96 | |
| | 90% CI for GM Ratio¹ | (0.64, 0.944) | (0.769, 1.134) | (0.793, 1.169) | |
| AUCinf(ng-h/mL) | N | 11 | 11 | 11 | 11 |
| | AM ± SD | 97.26 ± 52.22 | 111.17 ± 68.99 | 112.82 ± 55.95 | 114.04 ± 52.82 |
| | GM | 84.40 | 100.75 | 105.61 | 107.21 |
| | CV % | 53.7% | 62.1% | 49.6% | 46.3% |
| | Range | 10.69-200.53 | 42.36-270.98 | 45.89-221.97 | 39.39-192.58 |
| | GM Ratio¹ | 0.78 | 0.93 | 0.98 | |
| | 90% CI for GM Ratio¹ | (0.652, 0.95) | (0.778, 1.135) | (0.816, 1.189) | |
| Cmax(ng/mL) | N | 11 | 11 | 11 11 | |
| | AM ± SD | 7.08 ± 3.45 | 8.73 ± 4.19 | 10.55 ± 7.65 | 19.72 ± 7.47 |
| | GM | 6.41 | 8.08 | 9.06 | 18.58 |
| | CV % | 48.7% | 47.9% | 72.5% | 37.9% |
| | Range | 2.74-13.12 | 3.91-16.81 | 3.69-30.59 | 8.65-29.07 |
| | GM Ratio¹ | 0.34 | 0.43 | 0.48 | |
| | 90% CI for GM Ratio¹ | (0.267, 0.445) | (0.337, 0.561) | (0.378, 0.629) | |

AUCtlast = Area under the concentration-time curve C(t) from time zero to the last measurable sampling time point, t
AUCinf = Area under concentration-time curve from time zero to infinity
Cmax = Maximum observed post-dose concentration
AM = Arithmetic mean, SD = Standard Deviation, GM = Geometric Mean, CV = Coefficient of Variation
¹Ratio of the test formulation to the reference formulation

TABLE 24b
Descriptive Statistics for Pharmacokinetic Parameters (Tmax, Kel, t½)
by Treatment under Fed Conditions

| Pk Parameter | Statistics | Treatment A | Treatment B | Treatment C | Treatment D |
|---|---|---|---|---|---|
| Tmax | N | 11 | 11 | 11 | 11 |
| | AM ± SD | 10.45 ± 6.27 | 7.18 ± 2.60 | 7.27 ± 2.97 | 2.27 ± 0.61 |
| | CV % | 59.9% | 36.2% | 40.8% | 26.7% |
| | Median | 10.00 | 6.00 | 8.00 | 2.50 |
| Kel | N | 11 | 11 | 11 | 11 |
| | Range | 1.00-20.00 | 5.00-12.00 | 1.00-12.00 | 1.50-3.00 |
| | AM ± SD | 0.15 ± 0.04 | 0.16 ± 0.03 | 0.14 ± 0.04 | 0.16 ± 0.04 |
| | CV % | 26.8% | 19.4% | 28.7% | 26.0% |
| | Median | 0.15 | 0.16 | 0.15 | 0.16 |
| | Range | 0.10-0.24 | 0.11-0.20 | 0.04-0.18 | 0.12-0.27 |
| T½ | N | 11 | 11 | 11 | 11 |
| | AM ± SD | 4.77 ± 1.21 | 4.51 ± 0.95 | 6.09 ± 4.44 | 4.49 ± 1.00 |
| | CV % | 25.4% | 20.9% | 72.9% | 22.3% |
| | Median | 4.71 | 4.36 | 4.59 | 4.29 |
| | Range | 2.83-7.18 | 3.44-6.05 | 3.87-19.28 | 2.59-6.02 |

Tmax = time to attain Cmax
Kel = apparent elimination rate constant determined by log-linear regression of the terminal log-linear segment of the plasma concentration-time curve
t½ = apparent elimination half-life calculated as 0.693/Kel
SD = Standard Deviation, CV = Coefficient of Variation

### EXAMPLE 23

In Example 23, placebo tablets without the active (metoprolol tartrate) were prepared with a sustained release excipient of Example 1. The tablets were prepared in accordance with the process of Examples 2-5 (replacing metoprolol tartrate with mannitol) and having the formula listed in the table below:

TABLE 25

| | Example 23 |
|---|---|
| Component | (mg/tab) |
| Sustained Release Excipient of | 297.0 |
| Example 1 | |
| Metoprolol Tartrate | — |
| Mannitol | 50 |
| Hydroxypropylmethylcellulose | 15.5 |
| Talc | 19.7 |
| Sodium stearyl fumarate | 5.8 |
| TOTAL | 388.0 |

### EXAMPLES 24-26

In Examples 24-26, clinical batches of sustained release tablets prepared in accordance with Examples 4, 5, and 23 were coated with a sustained release coating (80:20 Surelease/Opadry ratio) to a 11% coating level and providing the formulas listed in the table below:

TABLE 26

| | Example 24 | Example 25 | Example 26 |
|---|---|---|---|
| Component | (mg/tab) | (mg/tab) | (mg/tab) |
| Example 4 tablet | — | 388 | — |
| Example 5 tablet | 388 | — | — |
| Example 23 tablet | — | — | 388 |
| Surelease | 34 | 34 | 34 |
| Opadry II Clear | 9 | 9 | 9 |
| TOTAL | 431 | 431 | 431 |

Process:

1. Add requisite amount Opadry II Clear to water and stir until solution is formed.

2. Add the requisite amount of Surelease to Opadry II Clear solution and stir for one hour.

3. Pass the Surelease/Opadry II Clear dispersion through a #20 mesh screen into a stainless steel container with a mixer.

4. Using a vector LDCS coating unit, coat the tablets with a Surelease/Opadry II clear 80/20 ratio dispersion to a target weight gain using the following settings:

| | |
|---|---|
| a. Inlet air temp.: | 55-75° C. |
| b. Exhaust air temp.: | 25-40° C. |
| c. Air volume: | 20-40 cfm |
| d. Pan rotation: | 20-30 rpm |

5. After the coating is complete, dry the tablets for 15-20 minutes (bed temp 40° C.) and allow the tablets to cool to approximately room temperature before discharging.

The coated tablets prepared in accordance with Examples 24-26 were further coated with a color coating of Opadry II Blue to a 3% coating level and having the formulas listed in the table below:

TABLE 27

| | Example 24 | Example 25 | Example 26 |
|---|---|---|---|
| | (mg/tab) with | (mg/tab) with | (mg/tab) with |
| Component | color coat | color coat | color coat |
| Example 24 | 431 | — | — |
| Example 25 | — | 431 | — |
| Example 26 | — | — | 431 |
| Opadry II Blue | 13 | 13 | 13 |
| TOTAL | 444 | 444 | 444 |

Process:

1. Prepare a suspension of 15% solids of Opadry II Blue.

2. Using a vector LDCS coating unit, coat the tablets with the Opadry II Blue suspension to the target weight gain using the following settings:

| | |
|---|---|
| a. Inlet air temp.: | 55-75° C. |
| b. Exhaust air temp.: | 25-40° C. |
| c. Air volume: | 20-40 cfm |
| d. Pan rotation: | 20-30 rpm |

After the coating is complete, dry the tablets for 15-20 minutes (bed temp 40° C.) and allow the tablets to cool to approximately room temperature before discharging.

### EXAMPLE 27

Tablets having formulation of Examples 4, 5, 24 and 25 were dissolution tested using USP dissolution Apparatus Type III in 250 ml of 0.1 M pH 7.5, at 15 dpm, and gave the results listed in Table 28:

TABLE 28

| | | | Example 24 | | | Example 24 |
|---|---|---|---|---|---|---|
| | | | with color | | | with color |
| Time | Example 5 | Example 24 | coat | Example 4 | Example 25 | coat |
| (hr) | % dissolved | % dissolved | % dissolved | % dissolved | % dissolved | % dissolved |
| 0 | 0.0 | 0.0 | 0.0 | 0.0 | 0.0 | 0.0 |
| 1 | 25.8 | 1.0 | 1.4 | 27.1 | 1.7 | 0.9 |
| 3 | 47.8 | 15.9 | 16.2 | 51.6 | 17.9 | 14.6 |
| 6 | 70.8 | 36.7 | 36.8 | 76.2 | 40.6 | 36.5 |
| 8 | 82.7 | 49.0 | 48.5 | 87.5 | 54.7 | 48.9 |
| 16 | 100.4 | 81.6 | 79.5 | 104.0 | 88.1 | 80.1 |
| 24 | 101.4 | 98.3 | 95.8 | 105.2 | 100.7 | 93.6 |
| Rem- | 0.2 | 3.2 | | 0.1 | 1.8 | |
| nant | | | | | | |
| Total | 101.5 | 101.4 | | 105.3 | 102.6 | |

### EXAMPLE 28

In Example 28 a randomized, single-blind, parallel group pilot study to compare the pharmacokinetics and pharmacodynamics of metoprolol tartrate sustained release tablets of Examples 24 (color coated) and 25 (color coated) to placebo of Example 26 (color coated). Forty healthy subjects (20 male, 20 female) were enrolled in the study. The participants were randomized to receive treatment as follows: 16 subjects received the sustained release metoprolol tartrate 25 mg tablets of Example 24 (color coated), 16 subjects received the sustained release metoprolol tartrate 50 mg tablets of Example 25 (color coated), and 8 subjects received placebo of Example 26 (color coated). The tablets were administered orally for the indication of Beta₁-adrenergic receptor antagonist. Each subject underwent conditioning exercise tests on Day 1 and 2 and received sustained release metoprolol tartrate tablets (placebo, 25 mg, or 50 mg) once daily on Days 3 through 7. Steady-state pharmacokinetics and pharmacodynamics were assessed on Days 7 and 8. Exercise tests for determination of metoprolol pharmacokinetics and pharmacodynamics were conducted on Days 1 and 2 (conditioning exercise tests), Day 3 at pre-dose (baseline exercise test), and Day 7 at pre-dose and 1, 2, 4, 8, 12, and 24 hours after dose administration (steady-state exercise tests). Blood samples for determination of metoprolol pharmacokinetics were collected on Day 3 prior to baseline exercise testing, pre-dose on Days 5 and 6 (trough samples), pre-dose and Day 7 at pre-dose and 1, 2, 4, 8, 12, and 24 hours after dose administration (steady-state samples).

Table 29 below lists the mean exercise heart rate (bpm) at baseline and during steady-state exercise tests.

TABLE 29

| | 25 mg | 50 mg | Placebo |
|---|---|---|---|
| Time (hr) | Mean | Mean | Mean |
| Baseline | 165 | 164.0 | 160 |
| 0 | 153 | 147 | 153 |
| (pre-dose) | | | |
| 1 | 152 | 148 | 159 |
| 2 | 151 | 145 | 153 |
| 4 | 149 | 139 | 153 |
| 8 | 145 | 138 | 151 |
| 12 | 150 | 146 | 154 |
| 24 | 147 | 144 | 142 |

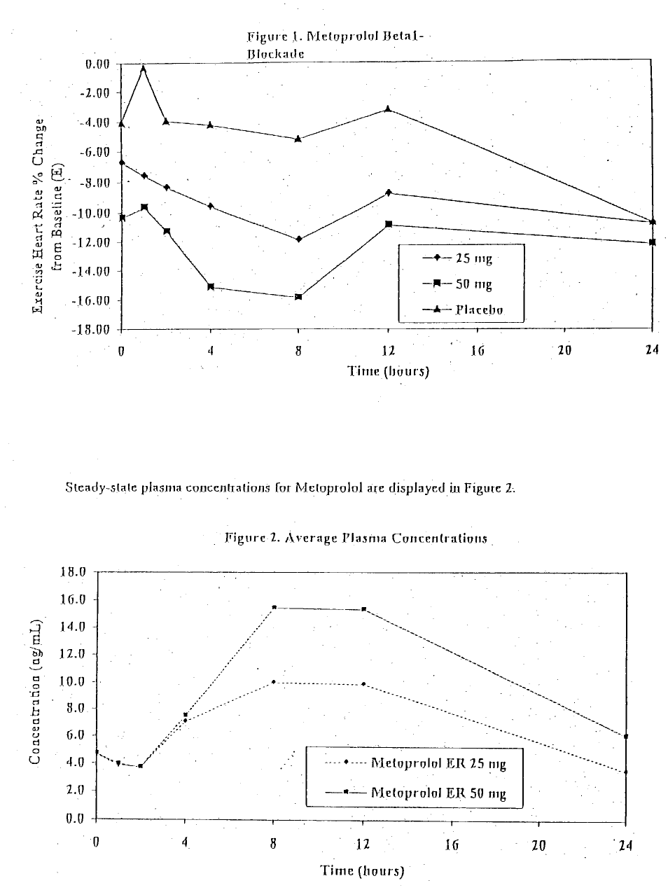

Though the placebo group appeared to have a slightly lower baseline exercise rate than the two active treatment, Beta₁-blockade is expressed as a percent change from baseline in exercise heart rate. Metoprolol Beta₁-blockade (E) is displayed in Table 30 below and in FIG. 1. The change from baseline in exercise heart rate (Beta₁-blockade) in both the active treatment was larger than the placebo from times 0 to 12 hours at steady state. At the 24-hour time point, the placebo treatment group had a significant fall in exercise heart rate while the exercise heart rate was maintained from 12 hours for the active treatment. Beta₁-blockade was evident in both active treatment groups throughout the 24-hour interval.

TABLE 30

| Time (hr) | 25 mg | 50 mg | Placebo |
|---|---|---|---|
| 0 | −6.8 | −10.4 | −4.1 |
| 1 | −7.6 | −9.7 | −0.4 |
| 2 | −8.4 | −11.3 | −4.0 |
| 4 | −9.6 | −15.1 | −4.3 |
| 8 | −11.9 | −15.8 | −5.2 |
| 12 | −9.2 | −10.9 | −3.3 |
| 24 | −11.1 | −12.3 | −10.8 |
| 0-12 hr average | −8.9 | −12.3 | −3.5 |
| 0-24 hr average | −9.2 | −12.1 | −5.0 |

Table 31 below displays the following pharmacodynamic variables for each of the treatment groups:

| | | |
|---|---|---|
| 1. Emin | = | Effect at time = 0 value on Day 7 |
| 2. Emax | = | Maximum effect following dose administration |
| 3. AUECss | = | Area under the Et (effect at time t) versus time curve |
| | | for one dosage interval at steady-state. |

TABLE 31

| Variable | 25 mg | 50 mg | Placebo |
|---|---|---|---|
| 0-12 hours | | | |
| Emin | −6.8 | −10.4 | −4.1 |
| Emax | −13.8 | −17.7 | −10.6 |
| AUECss | −118.4 | −162.3 | −44.2 |
| 0-24 hours | | | |
| Emin | −6.8 | −10.4 | −4.1 |
| Emax | −15.2 | −18.4 | −12.0 |
| AUECss | −240.6 | −301.3 | −128.8 |

The steady-state plasma concentrations for the sustained release metoprolol tartrate 25 mg and 50 mg from the study of Example 28 are graphically represented in FIG. 2. In addition, the mean pharmacokinetic parameters (median value for Tₘₐₓ) during steady state exercises are listed in Table 32 below:

TABLE 32

| | 25 mg | | 50 mg | |
|---|---|---|---|---|
| Variable | | | | |
| AUCss (ng · hr/ml) | 175.41 | (143.32) | 254.61 | (212.83) |
| Cmin (ng/ml) | 4.77 | (7.15) | 10.61 | (8.87) |
| Cmax (ng/ml) | 11.29 | (8.95) | 17.24 | (12.76) |
| Cavg | 7.31 | (5.97) | 4.68 | (6.58) |
| Fluctuation Index | 1.16 | (0.44) | 1.33 | (0.44) |
| Tmax (hr) | 12.0 | (8-12) | 12.0 | (8-12) |
| Mean Trough Plasma | | | | |
| Concentrations (ng/ml) | | | | |
| Day 5 | 4.07 | | 5.89 | |
| Day 6 | 3.41 | | 3.52 | |
| Day 7 | 4.58 | | 4.68 | |

Cmin = Minimum plasma concentration following dose administration
Cmax = Maximum plasma concentration following dose administration
Cavg = Average plasma concentration following dose administration
Tmax = Time to achieve Cmax
AUCss = Area under the plasma concentration versus time curve for one dosage interval at steady-state

In conclusion, Beta₁-blockade was evident in both active treatment groups throughout the 24-hour interval. The results of the study indicate that metoprolol tartrate sustained release tablets as described herein at a dosage rate of 25 mg once daily produce measurable Beta₁-adrenergic blockade throughout the dosage interval at steady state. On average, the degree of Beta₁-adrenergic blockade following administration of the 25 mg dose appears to be approximately 75% of the degree of Beta₁-adrenergic blockade produced by the 50 mg dose.

### EXAMPLE 29

In Example 29, a sustained release metoprolol tartrate oral dosage form was prepared having the formulation in the table below:

TABLE 33

| Description | Wt % | mg/tablet | g/batch^{a} |
|---|---|---|---|
| Core | | | |
| Metoprolol tartrate, USP | 31.20 | 200.0 | 321.10 |
| Sustained Release Excipient of Example 1 | 46.33 | 297.0 | 476.81 |
| Hypromellose 2910, USP | 3.46 | 22.2 | 35.61 |
| Talc, NF | 4.29 | 27.5 | 44.15 |
| Sodium stearyl fumarate, NF | 2.17 | 13.9 | 22.33 |
| Sterile water for injection, USP | —^{b} | —^{b} | 320.49^{b} |
| TOTAL | 87.45 | 560.6 | 900.00 |
| Functional Coating | | | |
| Surelease ® E-7-7050 (based on 25% | 7.71 | 49.4 | 79.20 |
| solids) | | | |
| Opadry ® II Clear Y-19-7483 | 1.92 | 12.3 | 19.80 |
| Sterile water for injection, USP | —^{b} | —^{b} | 158.40^{b} |
| Color Coating | | | |
| Opadry ® II Blue (40C90577) | 2.92 | 18.7 | 29.98 |
| Sterile water for injection, USP | —^{b} | —^{b} | 119.92^{b} |
| TOTAL | 12.55 | 55.6 | 128.98 |
| FINAL TABLET TOTAL | 100.00 | 641.0 | 1028.98 |

^{a}1,605 tablets per batch
^{b}Not present in the finished product

Process:

1. Accurately weight all the ingredients.

2. Blend Metoprolol Tartrate, USP, and the Sustained Release Excipient and screen through a mesh to break large agglomerates.

3. Prepare the granulating solution of 10% w/w Hypromellose 2910 (Methocel® E-5LV), USP in water.

4. Spray granulate with Hypromellose 2910, USP solution and dry in a fluid bed dryer.

5. Screen/mill the resulting granulation using a vibratory sieve/mill.

6. Blend the granulation with Talc, USP and Sodium stearyl fumarate, NF in a blender and compress into tablets on a rotary tablet press.

7. Coat the core tablets in a pan coater with a functional combination coating of Surelease®/Opadry® II clear dispersion.

8. Coat the functional coating with a color coating using Opadry II color coating system.

### EXAMPLE 30

In Example 30, a sustained release metoprolol tartrate oral dosage form was prepared having the formulation in the table below:

TABLE 34

| Description | Wt % | mg/tablet | g/batch^{a} |
|---|---|---|---|
| Core | | | |
| Metoprolol tartrate, USP | 30.30 | 200.0 | 321.10 |
| Sustained Release Excipient of Example 1 | 44.99 | 297.0 | 476.81 |
| Hydroxypropyl methycellulose, USP | 3.36 | 22.2 | 35.61 |
| Talc, NF | 4.17 | 27.5 | 44.15 |
| Sodium stearyl fumarate, NF | 2.11 | 13.9 | 22.33 |
| Sterile water for injection, USP | —^{b} | —^{b} | 320.49^{b} |
| TOTAL | 84.93 | 560.6 | 900.00 |
| Functional Coating | | | |
| Opadry II Blue 85F90578 (HP) | 2.55 | 16.8 | 27.0 |
| Surelease ® E-7-7050 (based on 25% | 7.69 | 50.8 | 81.58 |
| solids) | | | |
| Opadry ® Clear YS-1-19025A | 1.92 | 12.7 | 20.39 |
| Sterile water for injection, USP | —^{b} | —^{b} | 262.40^{b} |
| Color Coating | | | |
| Opadry II-Blue 85 F90578 (HP) | 2.91 | 19.2 | 30.87 |
| Sterile water for injection, USP | —^{b} | —^{b} | 123.48 |
| TOTAL | 15.07 | 99.5 | 159.84 |
| FINAL TABLET TOTAL | 100.00 | 660.1 | 1059.84 |

^{a}1,605 tablets per batch
^{b}Not present in the finished product

Process:

The tablets of Example 30 were prepared using the process as in Example 29.

### EXAMPLE 31

In Example 31, a sustained release oral dosage form was prepared having the formulation in the table below:

TABLE 35

| Description | Wt % | mg/tablet | g/batch^{a} |
|---|---|---|---|
| Core | | | |
| Metoprolol tartrate, USP | 32.07 | 200.0 | 321.10 |
| Sustained Release Excipient of Example 1 | 47.62 | 297.0 | 476.81 |
| Hydroxypropyl methycellulose, USP | 3.56 | 22.2 | 35.61 |
| Talc, NF | 4.41 | 27.5 | 44.15 |
| Sodium stearyl fumarate, NF | 2.23 | 13.9 | 22.33 |
| Sterile water for injection, USP | —^{b} | —^{b} | 320.49^{b} |
| TOTAL | 89.89 | 560.6 | 900.00 |
| Functional Coating | | | |
| Eudragit ® RS30D (based on 30% solids) | 3.80 | 23.7 | 38.01 |
| Eudragit ® RL30D (based on 30% solids) | 0.96 | 6.0 | 9.62 |
| Triethyl citrate, NF | 0.96 | 6.0 | 9.62 |
| Silicon dioxide, NF | 1.44 | 9.0 | 14.43 |
| Simethicone, USP | 0.03 | 0.2 | 0.32 |
| Sterile water for injection, USP | —^{b} | —^{b} | 159.53^{b} |
| Color Coating | | | |
| Opadry II Blue (40C90577) | 2.92 | 18.2 | 29.16 |
| Sterile water for injection, USP | —^{b} | —^{b} | 116.64^{b} |
| TOTAL | 10.11 | 99.5 | 101.16 |
| FINAL TABLET TOTAL | 100.00 | 623.7 | 1001.16 |

^{c}1,605 tablets per batch
^{d}Not present in the finished product

Process:

1. Accurately weight all the ingredients.

2. Blend Metoprolol Tartrate, USP, and the Sustained Release Excipient and screen through a mesh to break large agglomerates.

3. Prepare the granulating solution of 10% w/w hydroxypropymethylcellulose, USP in water.

4. Spray granulate with hydroxypropylmethylcellulose, USP solution and dry in a fluid bed dryer.

5. Screen/mill the resulting granulation using a vibratory sieve/mill.

6. Blend the granulation with Talc, USP and Sodium stearyl fumarate, NF in a blender and compress into tablets on a rotary tablet press.

7. Coat the core tablets in a pan coater with a functional combination coating of Eudragit® RS30D/RL30D.

8. Coat the functional coating with a color coating using Opadry II color coating system.

Many other variations of the present invention will be apparent to those skilled in the art and are meant to be within the scope of the claims appended hereto.

What is claimed is:

1. A sustained release oral solid dosage form comprising:
a sustained release matrix comprising from about 12.5 to about 400 mg of metoprolol tartrate and a sustained release excipient;
said oral dosage form providing a mean Cmax of metoprolol from about 10 ng/ml to about 40 ng/ml when the dosage form contains 100 mg metoprolol tartrate; said dosage form providing a therapeutic effect for about 24 hours after oral administration.

2. The sustained release oral dosage form of claim 1, wherein said mean Cmax of metoprolol is from about 15 ng/ml to about 30 ng/ml when the dosage form contains 100 mg metoprolol tartrate.

3. The sustained release oral dosage form of claim 1, wherein said oral dosage form provides a mean Cₘₐₓ of metoprolol from about 40 ng/ml to about 90 ng/ml when the dosage form contains 200 mg metoprolol tartrate.

4. The sustained release oral dosage form of claim 1, wherein said oral dosage form provides a mean Cₘₐₓ of metoprolol from about 5 ng/ml to about 30 ng/ml when the dosage form contains 50 mg metoprolol tartrate.

5. The sustained release oral dosage form of claim 1, wherein said oral dosage form provides a mean Cₘₐₓ of metoprolol from about 2 ng/ml to about 15 ng/ml when the dosage form contains 25 mg metoprolol tartrate.

6. The sustained release oral dosage form of claim 1, wherein said oral dosage form is substantially dose proportional.

7. The sustained release oral dosage form of claim 1, wherein said sustained release excipient is pre-agglomerated prior to incorporation of the metoprolol tartrate.

8. The sustained release oral dosage form of claim 1, wherein said dosage form provides a mean Tmax at from about 2.5 to about 20 hours after oral administration.

9. The sustained release oral dosage form of claim 1, wherein said dosage form provides a mean Tmax at from about 6 to about 16 hours after oral administration.

10. The sustained release oral dosage form of claim 1, wherein said sustained release matrix comprises a plurality of matrix multiparticulates.

11. The sustained release oral dosage form of claim 10, wherein said matrix multiparticulates are overcoated with a coating comprising a hydrophobic material.

12. The sustained release oral dosage form of claim 11, wherein said hydrophobic material is selected from the group consisting a hydrophobic polymer, a cellulosic material, an acrylic polymer, a methacrylic acid polymer, a methacrylic copolymer, hydrogenated vegetable oils, zein, and mixtures thereof.

13. The sustained release oral dosage form of claim 11, wherein said hydrophobic material is ethylcellulose.

14. The sustained release oral dosage form of claim 11, wherein said hydrophobic material comprises one or more ammonio methacrylate copolymers.

15. The sustained release oral dosage form of claim 1, wherein said dosage form is a compressed tablet.

16. The sustained release oral dosage form of claim 15, further comprising a coating over said compressed tablet; said coating comprising a hydrophobic material.

17. The sustained release oral dosage form of claim 16, wherein said hydrophobic material is selected from the group consisting of a hydrophobic polymer, a cellulosic material, an acrylic polymer, a methacrylic acid polymer, a methacrylic acid copolymer, hydrogenated vegetable oils, zein, and mixtures thereof.

18. The sustained release oral dosage form of claim 1, wherein the sustained release excipient comprises a gelling agent selected from the group consisting of a heteropolysaccharide gum, a homopolysaccharide gum, and mixtures thereof.

19. The sustained release oral dosage form of claim 1, wherein the sustained release excipient comprises a heteropolysaccharide gum and a homopolysaccharide gum.

20. The sustained release oral dosage form of claim 19, wherein said heteropolysaccharide gum is xanthan gum and said homopolysaccharide gum is locust bean gum.

21. The sustained release oral dosage form of claim 1, wherein the sustained release excipient further comprises a ionizable gel strength-enhancing agent.

22. The sustained release oral dosage form of claim 21, wherein said ionizable gel strength-enhancing agent is selected from the group consisting of calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride, and mixtures thereof.

23. A sustained release oral solid dosage form comprising:
a sustained release matrix comprising from about 12.5 to about 400 mg metoprolol tartrate and a sustained release excipient;
said oral dosage form providing a mean Cmax at steady state of metoprolol from about 4 ng/ml to about 20 ng/ml when the dosage form contains 25 mg metoprolol tartrate; said dosage form providing a therapeutic effect for about 24 hours after oral administration.

24. The sustained release oral dosage form of claim 23, wherein the Cmax at steady state of metoprolol is from about 6 ng/ml to about 15 ng/ml when the dosage form contains 25 mg metoprolol tartrate.

25. The sustained release oral dosage form of claim 23, wherein said dosage form provides a mean Tmax at from about 2.5 to about 20 hours after oral administration.

26. The sustained release oral dosage form of claim 23, wherein said dosage form provides a mean Tmax at from about 6 to about 16 hours after oral administration.

27. The sustained release oral dosage form of claim 23, wherein said sustained release matrix comprises a plurality of matrix multiparticulates.

28. The sustained release oral dosage form of claim 27, wherein said matrix multiparticulates are overcoated with a coating comprising a hydrophobic material.

29. The sustained release oral dosage form of claim 28, wherein said hydrophobic material is selected from the group consisting a hydrophobic polymer, a cellulosic material, an acrylic polymer, a methacrylic acid polymer, a methacrylic copolymer, hydrogenated vegetable oils, zein, and mixtures thereof.

30. The sustained release oral dosage form of claim 28, wherein said hydrophobic material is ethylcellulose.

31. The sustained release oral dosage form of claim 28, wherein said hydrophobic material comprises one or more ammonio methacrylate copolymers.

32. The sustained release oral dosage form of claim 23, wherein said dosage form is a compressed tablet.

33. The sustained release oral dosage form of claim 32, further comprising a coating over said compressed tablet; said coating comprising a hydrophobic material.

34. The sustained release oral dosage form of claim 32, wherein said hydrophobic material is selected from the group consisting of a hydrophobic polymer, a cellulosic material, an acrylic polymer, a methacrylic acid polymer, a methacrylic acid copolymer, hydrogenated vegetable oils, zein, and mixtures thereof.

35. The sustained release oral dosage form of claim 23, wherein the sustained release excipient comprises a gelling agent selected from the group consisting of a heteropolysaccharide gum, a homopolysaccharide gum, and mixtures thereof.

36. The sustained release oral dosage form of claim 23, wherein the sustained release excipient comprises a heteropolysaccharide gum and a homopolysaccharide gum.

37. The sustained release oral dosage form of claim 36, wherein said heteropolysaccharide gum is xanthan gum and said homopolysaccharide gum is locust bean gum.

38. The sustained release oral dosage form of claim 23, wherein the sustained release excipient further comprises a ionizable gel strength-enhancing agent.

39. The sustained release oral dosage form of claim 38, wherein said ionizable gel strength-enhancing agent is selected from the group consisting of calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride, and mixtures thereof.

40. A sustained release oral solid dosage form comprising:
a sustained release matrix comprising from about 12.5 mg to about 400 mg of metoprolol tartrate and a sustained release excipient; said oral dosage form providing a mean Cmax at steady state of metoprolol from about 5 ng/ml to about 30 ng/ml when the dosage form contains 50 mg metoprolol tartrate; said dosage form providing a therapeutic effect for about 24 hours after oral administration.

41. A sustained release oral dosage form comprising:
metoprolol tartrate in an amount of from about 12.5 mg to about 400 mg dispersed in a matrix comprising (i) a gelling agent said gelling agent in an amount of from about 10% to about 60% by weight of the dosage form, (ii) an inert pharmaceutical diluent in an amount of from about 5% to about 40% by weight of the dosage form, and (iii) an ionizable gel strength enhancing agent in an amount of from about 0.5% to about 16% by weight of the dosage form; a hydrophobic coating coated over said matrix in an amount of from about 1% to about 20% by weight of the dosage form; said dosage form providing for a sustained release of said metoprolol tartrate suitable for once a day administration.

42. The sustained release oral dosage form of claim 41, wherein said matrix is pre-agglomerated prior to incorporation of the metoprolol tartrate.

43. The sustained release oral dosage form of claim 41, wherein said gelling agent consists of a heteropolysaccharide gum and a homopolysaccharide gum.

44. The sustained release oral dosage form of claim 41, wherein said inert pharmaceutical diluent is mannitol.

45. A sustained release oral dosage form comprising:
a matrix comprising metoprolol tartrate in an amount of from about 12.5 mg to about 400 mg dispersed in a sustained release excipient comprising (i) locust bean gum in an amount of 5% to about 30% by weight of the oral dosage form and (ii) xanthan gum in an amount from about 5% to about 30% by weight of the oral dosage form, (iii) mannitol in an amount of from about 5% to about 40% by weight of the oral dosage form, and (iv) calcium sulfate dihydrate in an amount of about 0.5% to about 16% by weight of the oral dosage form; and
a hydrophobic coating coated over said matrix in an amount of from about 1% to about 20% by weight of the oral dosage form;
said dosage form providing for a sustained release of said metoprolol tartrate suitable for once-a-day administration.

46. A sustained release tablet formulation comprising:
a matrix core composition comprising
(a) metoprolol tartrate in an amount of from about 12.5 mg to about 400 mg;
(b) a cellulose derivative selected from the group consisting of an alkylcellulose, hydroxyalkylcellulose, hydroxypropylalkylcellulose, or mixtures thereof; and
(c) a sustained release excipient comprising a gelling agent in an amount of about 10% to about 60% by weight of the formulation; an inert diluent in an amount of from about 5% to about 40% by weight of the formulation; and an ionizable gel strength enhancing agent in an amount of from about 0.5% to about 16% by weight of the formulation; and
a coating over said core comprising
(a) a hydrophobic material in an amount of from about 2% to about 15% by weight of the formulation;
said formulation providing for a sustained release of said metoprolol tartrate suitable for once-a-day administration.

47. The sustained release tablet formulation of claim 46, wherein said hydrophobic material is selected from the group consisting of a hydrophobic polymer, a cellulosic material, an acrylic polymer, a methacrylic acid polymer, a methacrylic acid copolymer, hydrogenated vegetable oils, zein, and mixtures thereof

48. The sustained release tablet formulation of claim 46, wherein said hydrophobic material is ethylcellulose.

49. The sustained release tablet formulation of claim 46, wherein said hydrophobic material comprises one or more ammonio methacrylate copolymers.

50. The sustained release formulation of claim 46, wherein said coating further comprises hydroxypropylmethylcellulose.

51. A sustained release excipient for the sustained release of an active agent comprising from about 20% to about 60% of a gelling agent by weight of said sustained release excipient, said gelling agent consisting of a heteropolysaccharide gum and a homopolysaccharide gum; from about 1% to about 20% of an ionizable gel strength enhancing agent by weight of said sustained release excipient; and from about 6% to about 60% of mannitol by weight of the sustained release excipient.

52. The sustained release excipient of claim 51, further comprising a cellulosic material selected from the group consisting of alkylcellulose, hydroxyalkylcellulose, hydroxypropylalkylcellulose, or mixtures thereof.

53. The sustained release excipient of claim 51, wherein said heteropolysaccharide gum is xanthan gum.

54. The sustained release excipient of claim 51, wherein said homopolysaccharide gum is locust gum.

55. The sustained release excipient of claim 51, wherein said ionizable gel strength enhancing agent is selected from the group consisting of calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride, and mixtures thereof.

56. The sustained release excipient of claim 51, wherein said ionizable gel strength enhancing agent is calcium sulfate dihydrate.

57. A method of treating a patient with hypertension comprising:
administering to said patient a sustained release oral dosage form comprising a sustained release matrix comprising from about 12.5 to about 400 mg of metoprolol tartrate and a sustained release excipient; said oral dosage form providing a mean Cmax of metoprolol from about 10 ng/ml to about 40 ng/ml when the dosage form contains 100 mg metoprolol tartrate; said dosage form providing a therapeutic effect for about 24 hours after oral administration.

58. The method of claim 57, wherein said mean Cmax of metoprolol is from about 15 ng/ml to about 30 ng/ml when the dosage form contains 100 mg metoprolol tartrate.

59. A method of reducing blood pressure comprising
administering to a human patient a sustained release oral solid dosage form comprising a sustained release matrix comprising about 12.5 to about 400 mg of metoprolol tartrate and a sustained release excipient; said oral dosage form providing a mean Cmax of metoprolol from about 10 ng/ml to about 40 ng/ml when the dosage form contains 100 mg metoprolol tartrate, said dosage form providing a therapeutic effect for about 24 hours after oral administration.

60. A method of providing cardioselective antihypertensive therapy to a human patient comprising administering to said patient a sustained release oral dosage form comprising a sustained release matrix comprising about 12.5 to about 400 mg of metoprolol tartrate and a sustained release excipient; said oral dosage form providing a mean Cmax of metoprolol from about 10 ng/ml to about 40 ng/ml when the dosage form contains 100 mg metoprolol tartrate; said dosage form providing a therapeutic effect for about 24 hours after oral administration.

61. A method of preventing or reducing a mallaird-type reaction in a metoprolol tartrate sustained release oral dosage form comprising preparing said sustained release oral dosage form by combining a therapeutically effective amount of metoprolol tartrate with a sustained release excipient that provides for the sustained release of said metoprolol tartrate, and including in said dosage form an effective amount of mannitol to prevent or reduce the degradation of said metoprolol tartrate.

62. The method of claim 61, wherein said mannitol is included in said sustained release excipient prior to combining said excipient with said metoprolol tartrate.

63. The method of claim 61, wherein said mannitol is incorporated into said dosage when said metoprolol tartrate and said sustained release excipient are combined.

64. A method of preparing a sustained release tablet formulation of metoprolol tartrate for once-a-day administration comprising:
spray granulating a sustained release excipient and metoprolol tartrate with a suspension or solution comprising a cellulose derivative selected from the group consisting of an alkylcellulose, hydroxyalkylcellulose, hydroxyalkylalkylcellulose, or mixtures thereof; and tableting the resultant granulation such that each tablet provides a dose of metoprolol tartrate sufficient to provide a therapeutic effect for about 24 hours after oral administration.

65. The method of claim 64, further comprising overcoating the tablets with a coating comprising a hydrophobic material.

66. The method of claim 65, wherein the hydrophobic material is selected from the group consisting of hydrophobic polymer, a cellulosic material, an acrylic polymer, a methacrylic acid polymer, a methacrylic copolymer, hydrogenated vegetable oils, zein, and mixtures thereof.

67. A sustained release oral solid dosage form comprising:
a sustained release matrix comprising a therapeutically effective amount of metoprolol tartrate and a sustained release excipient;
said oral dosage form providing a mean Cmax of metoprolol from about 10 ng/ml to about 40 ng/ml per 100 mg metoprolol tartrate, said dosage form providing a therapeutic effect for about 24 hours after oral administration.

68. A method of treating a patient with hypertension comprising:
administering to said patient a sustained release oral dosage form comprising a sustained release matrix comprising a therapeutically effective amount of metoprolol tartrate and a sustained release excipient; said oral dosage form providing a mean Cmax of metoprolol from about 10 ng/ml to about 40 ng/ml per 100 mg metoprolol tartrate; said dosage form providing a therapeutic effect for about 24 hours after oral administration.

69. A method of reducing blood pressure comprising administering a sustained release oral dosage form of claim 67 to a human patient.

70. A method of reducing blood pressure comprising administering a sustained release oral dosage form of claim 67 to a human subject.

71. A method of providing cardioselective antihypertensive therapy to a human patient comprising administering a sustained release oral dosage form of claim 67 to a patient in need of said therapy.

72. A sustained release oral solid dosage form comprising:
a sustained release matrix comprising a therapeutically effective amount of metoprolol tartrate and a sustained release excipient; said oral dosage form providing a mean Cmax at steady state of metoprolol from about 5 ng/ml to about 30 ng/ml per 50 mg metoprolol tartrate, said dosage form providing a therapeutic effect for about 24 hours after oral administration.

73. A sustained release oral solid dosage form comprising:
a sustained release matrix comprising a therapeutically effective amount of metoprolol tartrate and a sustained release excipient;
said oral dosage form providing a mean Cmax at steady state of metoprolol from about 4 ng/ml to about 20 ng/ml per 25 mg metoprolol tartrate, said dosage form providing a therapeutic effect for about 24 hours after oral administration.

74. A sustained release tablet formulation comprising:
about 31% by weight of metoprolol tartrate;
about 45% by weight of a sustained release excipient comprising xanthan gum, locust bean gum, calcium sulfate dihydrate, and mannitol;
about 3% by weight hydroxypropylmethylcellulose;
about 4% by weight talc;
about 2% by weight sodium stearyl fumarate;
about 9 to about 12% by weight hydrophobic coating material; and
about 3% by weight of a color coating material;
said formulation providing for the sustained release of said metoprolol tartrate.

75. The sustained release tablet formulation of claim 74, wherein said hydrophobic coating material comprises ethylcellulose in an amount of about 8% by weight of the formulation.

76. The sustained release tablet formulation of claim 74, wherein said sustained release excipient comprises about 20% by weight xanthan gum; about 30% by weight locust bean gum; about 10% by weight calcium sulfate dihydrate; and about 40% by weight mannitol.

77. A sustained release tablet formulation comprising:
about 32% by weight of metoprolol tartrate;
about 48% by weight of a sustained release excipient comprising xanthan gum, locust bean gum, calcium sulfate dihydrate, and mannitol;
about 4% by weight hydroxypropylmethylcellulose;
about 4% by weight talc;
about 2% by weight sodium stearyl fumarate;
about 8% by weight hydrophobic coating material; and
about 3% by weight of a color coating material;
said formulation providing for the sustained release of said metoprolol tartrate.

78. The sustained release tablet formulation of claim 77, wherein said hydrophobic coating material comprises a combination of two ammonio methacrylate copolymers in a combined amount of about 5% by weight of the formulation.

79. The sustained release tablet formulation of claim 77, wherein said sustained release excipient comprises about 20% by weight xanthan gum; about 30% by weight locust bean gum; about 10% by weight calcium sulfate dihydrate; and about 40% by weight mannitol.
